(19) United States
(12) Patent Application Publication
(10) Pub. No.: US 2024/0376098 A1
(43) Pub. Date: Nov. 14, 2024

(54) METHODS OF TREATMENT USING BCN057, BCN077 AND ANALOGS

(71) Applicant: BCN Biosciences L.L.C., Inglewood, CA (US)

(72) Inventors: Andrew J. Norris, Los Angeles, CA (US); Elizabeth M. Singer, Los Angeles, CA (US); Sudip Chakrabortty, Los Angeles, CA (US)

(73) Assignee: BCN Biosciences L.L.C., Inglewood, CA (US)

(21) Appl. No.: 18/595,355

(22) Filed: Mar. 4, 2024

Related U.S. Application Data
(63) Continuation-in-part of application No. PCT/US2022/042662, filed on Sep. 6, 2022.
(60) Provisional application No. 63/240,847, filed on Sep. 3, 2021.

(51) Int. Cl. C07D 471/04; A61K 31/4709; A61K 45/06; A61P 35/00

(52) U.S. Cl. CPC C07D 471/04; A61K 31/4709; A61K 45/06; A61P 35/00

(57) ABSTRACT

The present disclosure is directed to methods of treating or ameliorating various conditions by the administration of a BCN057, BCN077 and analogs. BCN057, BCN077 and analogs can be used to modulate PD-1 and treat various ailments in which PD-1 expression is involved. The compounds have antineoplastic activity. Further, the compounds also have cytoprotective activity as they protect against chemotherapy induced toxicity to the GI tract. The compounds disclosed herein can be used to reduce tumor burden in cancers, including pancreatic cancer, gastrointestinal (GI) cancer and epithelial cancers.

### RELATED APPLICATIONS

This application is a Continuation in Part of International Application Number PCT/US2022/042662, filed Sep. 6, 2022, and claims priority to U.S. provisional patent application Ser. No. 63/240,847 filed on Sep. 3, 2021, the contents of which are incorporated herein by reference.

### FIELD OF THE INVENTION

The invention relates to small molecules for therapeutic use, and more specifically, to therapeutic applications of the BCN057, BCN077 and analogs thereof.

### BACKGROUND

Cancer can be defined as a group of diseases that involve abnormal cell growth with the potential to invade or spread to other parts of the body. Despite advances in technology, cancer continues to be a significant cause of death and incalculable suffering. Cancer is the second most common cause of death in the United States.

Patients with cancer often have limited treatment options. Treatment can include a combination of surgery, radiation therapy, chemotherapy and targeted therapy. Despite advances in research, these treatments remained relatively unchanged in recent decades. Efforts have often focused on early diagnosis of cancer when treatments are more effective. However, the survival times afforded by early diagnosis are modest.

Pancreatic cancer Pancreatic cancer arises when cells in the pancreas, a glandular organ behind the stomach, begin to multiply out of control and form a mass. These cancerous cells have the ability to invade other parts of the body. A number of types of pancreatic cancer are known. However, the most common, pancreatic adenocarcinoma, accounts for about 90% of cases. Signs and symptoms of the most-common form of pancreatic cancer may include yellow skin, abdominal or back pain, unexplained weight loss, light-colored stools, dark urine, and loss of appetite. Usually, no symptoms are seen in early stages of the disease. Symptoms that are specific enough to suggest pancreatic cancer usually do not develop until the disease has reached an advanced stage. By the time of diagnosis, pancreatic cancer has often spread to other parts of the body.

Chemotherapy is a type of cancer treatment that uses one or more anti-cancer drugs (chemotherapeutic agents) as part of a standardized chemotherapy regimen. Traditional chemotherapeutic agents are cytotoxic by means of interfering with cell division. However, cancer cells vary in their susceptibility to these agents. To a large extent, chemotherapy can be thought of as a way to damage or stress cells, which may then lead to cell death if apoptosis is initiated. Many of the side effects of chemotherapy can be traced to damage to normal cells that divide rapidly and are thus sensitive to anti-mitotic drugs. This results in the most common side-effects of chemotherapy: myelosuppression, mucositis, and alopecia. Because of the effect on immune cells (especially lymphocytes), chemotherapy drugs often find use in a host of diseases that result from harmful overactivity of the immune system against self (so-called autoimmunity).

Immune checkpoint inhibitory receptors (e.g., CTLA4 and PD-1) expressed on immune cells trigger immunosuppressive signaling pathways. CTLA4 competes with CD28 for binding to CD80 and CD86. PD-1 binds to PD-L1 or PD-L2 and resists positive signals through T-cell receptors (TCRs) and CD28. These immunosuppressive molecules function as brakes to regulate the adaptive immune response. Suppressive signals can be used in a variety of ways to maintain the balance of the immune system. Based on this application, the PD-1/PD-L pathway has stood out as an immune checkpoint. In general, expression of PD-L1 is observed on T, B, and antigen-presenting cells and in some non-lymphoid tissues.

The identification of tumor-specific antigens, lymphocytes, and T-cell responses in cancer patients led to immunotherapies designed to enhance antitumor immune responses. Biological products or “biologics” includes recombinant therapeutic proteins such as monoclonal antibodies (MABs). Biologic drugs can stimulate the body to spot cancer cells and attack them. Some biologics attack cancer cells directly and interfere with growth signals. Other biologics can help patients fight infection after chemotherapy.

T cells are potent cellular effectors of the immune system and have a memory that responds if rechallenged by the same antigen. Because tumor immune therapy is more specific and less toxic than traditional chemotherapy, it may be an ideal adjuvant treatment for patients with malignancies that have a high risk of relapse. Chemotherapy could be used first to induce the active disease into remission, followed by tumor immune therapy via memory T cells. However, biologics also have limitations. Immunotherapies seem to be effective against small tumor burdens, but they are not capable of controlling large tumor masses. Moreover, patients with advanced disease have compromised immunity, which limits the effectiveness of immunotherapy. Therefore, it has been proposed that immunotherapy be used in the early stages of tumorigenesis. Further, biologics are expensive compared to other agents and they must be administered intravenously. Further, their relatively large size limits their function and half-life in the blood.

Recent studies have provided new opportunities for cancer treatment, including the use of small molecules. Particular small molecules bind biological micromolecules and act as effectors, thereby altering the activity or function of the target. They manifest a variety of biological functions or applications, serving as cell signaling molecules. For example, small molecule cancer drugs, have been successfully used to target the extracellular, cell surface ligand-binding receptors as well as the intracellular proteins, including anti-apoptotic proteins that play a key role in transducing downstream signaling for cell growth and metastasis promotion. Many small molecule drugs inhibit critical cancer targets such as serine/threonine/tyrosine kinases, matrix metalloproteinases (MMPs), heat shock proteins (HSPs), proteosome and other proteins important in signal transduction pathways. The small size of the molecules (<500 Da) also allows them to translocate through the plasma membrane. In principle, small molecule compounds can be developed to target any portion of a cell, regardless of the target's cellular location. Such drugs have good therapeutic potential as they can inhibit critical cancer targets.

Effective treatment to pancreatic cancer challenging due to resistance for radiation/chemotherapy and poor drug permeability. Moreover, treatment induced normal tissue toxicity, mainly to the duodenum and gastrointestinal epithelium is common and a dose limiting event. Gastrointestinal toxicity often results interruption, reduction or pre-mature withdrawal of anti-cancer therapy which is a significant factor impacting the overall survival of patients being treated. Therefore, there is an urgent need to develop therapeutic strategies to selectively sensitize tumor tissue without inducing normal tissue toxicity. An ideal treatment/therapy would target cancer cells and reduce harmful effects of chemotherapy.

### SUMMARY OF THE INVENTION

Compound BCN057 disclosed herein was previously described in U.S. patent application Ser. No. 13/813,923 and U.S. patent application Ser. No. 14/889,719. The present invention provides new molecules and analogs and new methods of use. BCN057 can also be referred to by its structural name: (3-[(Furan-2-ylmethyl)-amino]-2-(7-methoxy-2-oxo-1,2-dihydro-quinolin-3-yl)-6-methyl-imidazo[1,2-a]pyridin-1-ium) or YEL002. BCN057 and its analogs are useful for, among other therapies, treating or preventing inflammatory disease and for treating or preventing cancer or other hyperproliferative conditions.

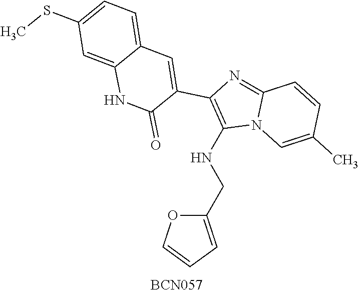

Applicants propose the small molecule BCN057, BCN077 can modulate chemo-sensitivity of oncogenic RAS pancreatic cancer cells but conversely protects normal intestinal epithelium from off target toxicity from the same by ameliorating chemo-toxicity in Lgr5 positive intestinal stem cells thereby preserving barrier function. Further, it is demonstrated that BCN057 and BCN077 inhibit GSK3β and thereby induces a pro-apoptotic phosphorylation pattern on c-Jun in KRAS G12D mutant pancreatic cancer cells leading to the restoration of PTEN expression and consequent apoptosis which is a novel mechanistic observation for the oncogenic RAS phenotype. Lastly, concurrent with its GSK3P inhibition, BCN057 and BCN077 are small molecule inhibitors of PD-1 expression on human T-lymphocytes co cultured with human pancreatic cancer cells. In summary BCN057 AND BCN077 can promote synthetic lethality specifically to malignant cells and therefore should be considered to improve the therapeutic ratio in pancreatic and epithelial cancer treatment in conjunction with chemotherapy and radiation.

One embodiment is a compound of Formula I or an analog thereof,

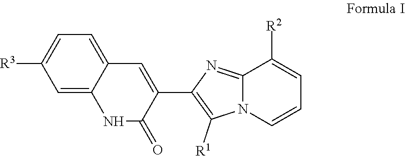

wherein R¹=an alkyl, an alkyl amine, an ether, an aryl or an aryl halide,
wherein R²=H or CH₃,
wherein R³=H, OH or OCH₃.

### Alternatively, R¹=

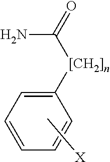

n=1, 2 or 3; X=F, CF₃ Cl, OH or CH₂OH; Y=O, S or N; and Z=CH₃, CH₂OH or OCH₃.

### Alternatively R¹=

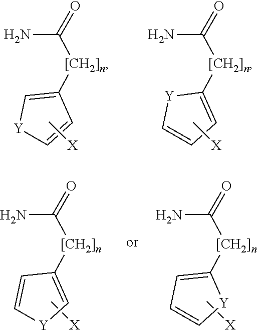

and wherein n=1, 2 or 3; X=F, CF₃ Cl, OH or CH₂OH; Y=O, S or N; and Z=CH₃, CH₂OH or OCH₃.

### Alternatively, R¹=

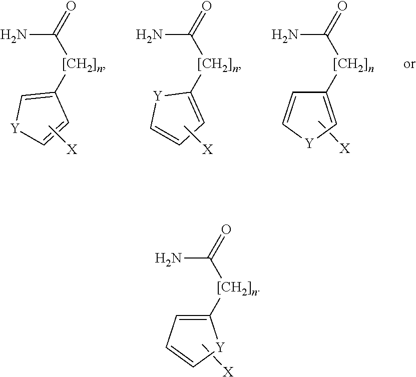

wherein n=1, 2 or 3, X=F, CF₃ Cl, OH or CH₂OH and Y=O, S or N.

### Alternatively, R¹=

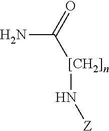

and n=1, 2 or 3.

One embodiment is a method of treating cancer comprising administering a therapeutic amount of the compound of Formula I or an analog thereof. The cancer can be, for example, bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, rectal cancer, skin cancer, blood cancer or testicular cancer.

The cancer can also be biliary tract cancer, bladder cancer, ganglia cancer (neuroblastoma), leukemias, lymphoma, liver cancer (e.g., hepatocellular carcinoma), lung cancer (e.g., large cell carcinoma, non-small cell carcinoma and squamous cell carcinoma), soft tissue cancer (e.g., angiosarcoma, leiomyosarcoma, liposarcoma, rhabdomyosarcoma, myxoma and malignant fibrous histiocytoma-pleomorphic sarcoma), stomach cancer or thyroid cancer.

Another embodiment is a method of modulating PD-1 expression to treat an ailment, comprising administering a therapeutic amount of the compound of Formula I or an analog thereof. The ailment can be, for example, cancer, cerebral malaria, Trypanosoma cruzi induced myocarditis, influenza virus A, tuberculosis bacillus, chlamydia lung infections, COPD, acute lung injury, HBV & HCV liver infection, pancreatitis, Type 1 diabetes, sepsis or HIV-1.

Another embodiment is a method of inhibiting PD-1, PD-L1 and/or the PD-1/PD-L1 interaction comprising administering an effective amount of the compound Formula I or an analog thereof. In aspects, the method blocks the interaction of PD-L1 with PD-1 and/or CD80. The method can include administration of an additional therapeutic agent, such as a small molecule, an antibody, an antibody fragment, an antibody conjugate or an immunomodulating agent. In aspects, the method includes administration of one or more immune checkpoint regulators.

Additional embodiments include a method of treating a viral infection in a subject in need thereof with a compound of Formula I or an analog thereof. Embodiments also include a method of treating depression with a compound of Formula I or an analog thereof.

Additional embodiments include a method of treating a viral infection in a subject in need thereof with a compound of Formula I or an analog thereof. Embodiments also include a method of treating depression with a compound of Formula I or an analog thereof.

One embodiment is a compound of Formula A. Additional embodiments include methods of treating an ailment in a subject, comprising administering to the subject a therapeutically effective amount of a compound having the structure of Formula A or an analog thereof,

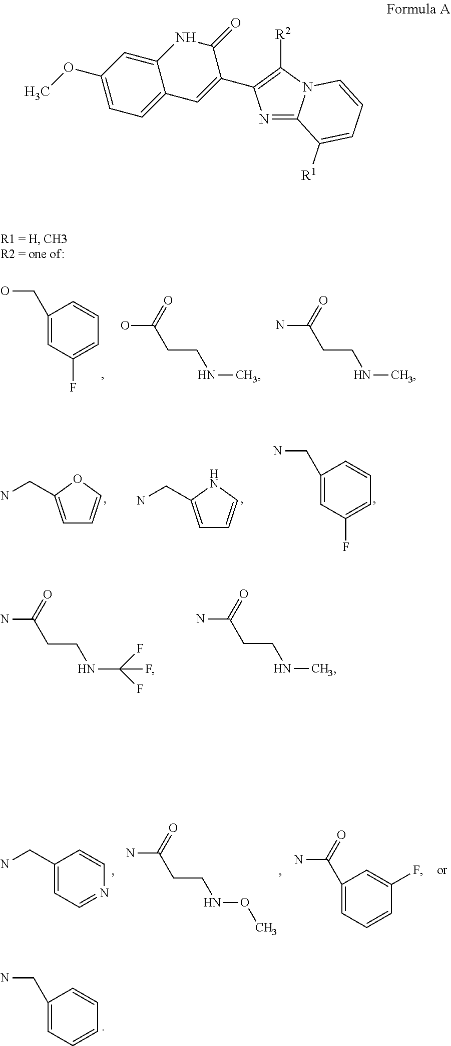

Additional embodiments include is a method of treating cancer in a subject with a therapeutically effective amount of a compound of Formula A or an analog thereof. The cancer can be bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, rectal cancer, skin cancer, blood cancer or testicular cancer. The treatment can include administering one or more additional medicaments to the subject as well as chemotherapy or radiotherapy.

Additional embodiments include a method of treating a subject with one or more side effects of chemotherapy or radiotherapy with the compound of Formula A or an analog thereof. Yet another embodiment is a method of preventing or treating radiation induced damage to epithelial cells by administering a therapeutically effective amount of a compound of Formula B or an analog thereof. The radiation induced damage to epithelial cells can be identified as radiation-induced gastrointestinal syndrome (RIGS), radiation-induced mucositis, radiation-induced oral mucositis, radiation-induced proctitis and/or radiation-induced enteritis.

Additional embodiments include a method of treating fibrosis with compound of Formula A or an analog thereof. The fibrosis can be pulmonary fibrosis, idiopathic pulmonary fibrosis, acute respiratory distress syndrome, cystic fibrosis, non-cystic fibrosis bronchiectasis, cirrhosis, liver fibrosis, endomyocardial fibrosis, old myocardial infarction, atrial fibrosis, mediastinal fibrosis, myelofibrosis, retroperitoneal fibrosis, progressive massive fibrosis, nephrogenic systemic fibrosis, Crohn's disease, gastrointestinal fibrosis, keloid conditions, scleroderma/systemic sclerosis, arthofibrosis, peyronie's disease, dupuytren's contracture, oral submucous fibrosis, liver fibrosis, gastrointestinal fibrosis, renal fibrosis from kidney dialysis and/or adhesive capsulitis.

Additional embodiments include a method of treating a viral infection in a subject in need thereof with a compound of Formula A or an analog thereof. Embodiments also include a method of treating depression with a compound of Formula B or an analog thereof.

Another embodiment is a method of inhibiting PD-1, PD-L1 and/or the PD-1/PD-L1 interaction comprising administering an effective amount of the compound Formula A or an analog thereof. In aspects, the method blocks the interaction of PD-L1 with PD-1 and/or CD80. The method can include administration of an additional therapeutic agent, such as a small molecule, an antibody, an antibody fragment, an antibody conjugate or an immunomodulating agent. In aspects, the method includes administration of one or more immune checkpoint regulators.

Another embodiment is a method of treating an ailment in a subject, comprising administering to the subject a therapeutically effective amount of a compound having the structure of Formula B:

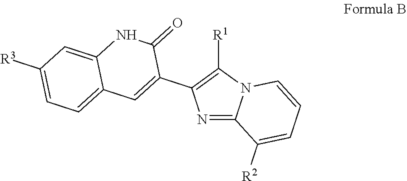

wherein:
R¹ is an alkyl, alkyl amine, or an ether;
R² is H or CH₃; and
R³ is H or OH.
   Analogs of Formula B can include the analogs detailed in Table 2, infra.

Additional embodiments include is a method of treating cancer in a subject with a therapeutically effective amount of a compound of Formula B or an analog thereof. The cancer can be bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, rectal cancer, skin cancer, blood cancer or testicular cancer. The treatment can include administering one or more additional medicaments to the subject as well as chemotherapy or radiotherapy.

Additional embodiments include a method of treating a subject with one or more side effects of chemotherapy or radiotherapy with the compound of Formula B or an analog thereof. Yet another embodiment is a method of preventing or treating radiation induced damage to epithelial cells by administering a therapeutically effective amount of a compound of Formula B or an analog thereof. The radiation induced damage to epithelial cells can be identified as radiation-induced gastrointestinal syndrome (RIGS), radiation-induced mucositis, radiation-induced oral mucositis, radiation-induced proctitis and/or radiation-induced enteritis.

Additional embodiments include a method of treating fibrosis with compound of Formula B or an analog thereof. The fibrosis can be pulmonary fibrosis, idiopathic pulmonary fibrosis, acute respiratory distress syndrome, cystic fibrosis, non-cystic fibrosis bronchiectasis, cirrhosis, liver fibrosis, endomyocardial fibrosis, old myocardial infarction, atrial fibrosis, mediastinal fibrosis, myelofibrosis, retroperitoneal fibrosis, progressive massive fibrosis, nephrogenic systemic fibrosis, Crohn's disease, gastrointestinal fibrosis, keloid conditions, scleroderma/systemic sclerosis, arthofibrosis, peyronie's disease, dupuytren's contracture, oral submucous fibrosis, liver fibrosis, gastrointestinal fibrosis, renal fibrosis from kidney dialysis and/or adhesive capsulitis.

Additional embodiments include a method of treating a viral infection in a subject in need thereof with a compound of Formula B or an analog thereof. Embodiments also include a method of treating depression with a compound of Formula B or an analog thereof.

Another embodiment is a method of treating an ailment in a subject comprising administering to the subject a therapeutically effective amount of a compound having the structure of Formula C:

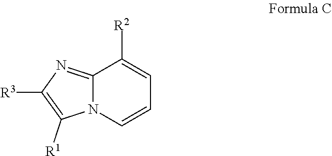

wherein R₃=

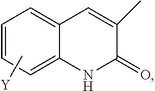

wherein Y=OCH₂CH₃, OCH(n)CH₃, NH₂, OH, OMe, Me, H, CH₂OH, BH₂, SeMe or SMe, and wherein R₁ and R₂ are independently hydrogen, straight chain or branched C1-C20 alkyl, alkenyl, alkynyl, which is substituted or unsubstituted, cyclo alkyl, cyclo alkenyl, hererocyclic alkyl, or heterocyclic alkenyl, which is substituted or unsubstituted, phenyl, substituted phenyl, aryl, substituted aryl, amino, amido, F, Cl, Br, I, nitro, hydroxyl, thiol, alkylthio, selenol, alkylselenyl, silyl, siloxy, boryl, carboxylic acid, sulfolyl, —SO₄H, alkoxy or acyl groups.

One embodiment is a method of treating cancer comprising administering a therapeutic amount of the compound of Formula C or an analog thereof. The cancer can be, for example, bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, rectal cancer, skin cancer, blood cancer or testicular cancer.

The cancer can also be biliary tract cancer, bladder cancer, ganglia cancer (neuroblastoma), leukemias, lymphoma, liver cancer (e.g., hepatocellular carcinoma), lung cancer (e.g., large cell carcinoma, non-small cell carcinoma and squamous cell carcinoma), soft tissue cancer (e.g., angiosarcoma, leiomyosarcoma, liposarcoma, rhabdomyosarcoma, myxoma and malignant fibrous histiocytoma-pleomorphic sarcoma), stomach cancer or thyroid cancer.

Another embodiment is a method of modulating PD-1 expression to treat an ailment, comprising administering a therapeutic amount of the compound of Formula C or an analog thereof. The ailment can be, for example, cancer, cerebral malaria, Trypanosoma cruzi induced myocarditis, influenza virus A, tuberculosis bacillus, chlamydia lung infections, COPD, acute lung injury, HBV & HCV liver infection, pancreatitis, Type 1 diabetes, sepsis or HIV-1.

Additional embodiments include a method of treating a viral infection in a subject in need thereof with a compound of Formula C or an analog thereof. Embodiments also include a method of treating depression with a compound of Formula C or an analog thereof.

Additional embodiments include a method of treating a viral infection in a subject in need thereof with a compound of Formula C or an analog thereof. Embodiments also include a method of treating depression with a compound of Formula I or an analog thereof.

Another embodiment is a method of inhibiting PD-1, PD-L1 and/or the PD-1/PD-L1 interaction comprising administering an effective amount of the compound Formula C or an analog thereof. In aspects, the method blocks the interaction of PD-L1 with PD-1 and/or CD80. The method can include administration of an additional therapeutic agent, such as a small molecule, an antibody, an antibody fragment, an antibody conjugate or an immunomodulating agent. In aspects, the method includes administration of one or more immune checkpoint regulators.

In another embodiment, the disclosure provides a method of treating cancer in a subject in need thereof, the method comprising the step of administering to the subject a therapeutically effective amount of a compound of BCN057 AND BCN077, or an analog thereof. In another embodiment, the disclosure provides a method of treating cancer in a subject in need thereof, the method comprising the step of administering to the subject a combination of medicaments that includes a therapeutically effective amount of compound BCN057 AND BCN077, or an analog thereof.

In another embodiment, the disclosure provides a method of treating side effects of chemotherapy or radiation therapy in a subject, the method comprising the step of administering to the subject a therapeutically effective amount of a compound of BCN057 AND BCN077, or an analog thereof. In another embodiment, the disclosure provides a method of treating radiation-induced gastrointestinal syndrome (RIGS) in a subject, the method comprising administering to the subject a therapeutically effective amount of compound BCN057 AND BCN077, or an analog thereof.

Another embodiment is a method blocking the interaction of PD-L1 with PD-1 and/or CD80 in a subject, the method comprising administering to the subject a therapeutically effective amount of the compound of Formula C, or an analog thereof.

Another embodiment is a method of inhibiting LAG-3 by administering a compound described herein, Without being bound by theory, the molecules described herein can inhibit GSK3β (the mechanism by which LAG-3 expression is inhibited in T-cells).

In certain embodiments, compounds of the invention may be prodrugs of the compounds of formula I or II, e.g., wherein a hydroxyl in the parent compound is presented as an ester or a carbonate, or carboxylic acid present in the parent compound is presented as an ester. In certain such embodiments, the prodrug is metabolized to the active parent compound in vivo (e.g., the ester is hydrolyzed to the corresponding hydroxyl, or carboxylic acid).

In certain embodiments, compounds of the invention may be racemic. In certain embodiments, compounds of the invention may be enriched in one enantiomer. For example, a compound of the invention may have greater than 30% ee, 40% ee, 50% ee, 60% ee, 70% ee, 80% ee, 90% ee, or even 95% or greater ee. In certain embodiments, compounds of the invention may have more than one stereocenter. In certain such embodiments, compounds of the invention may be enriched in one or more diastereomer. For example, a compound of the invention may have greater than 30% de, 40% de, 50% de, 60% de, 70% de, 80% de, 90% de, or even 95% or greater de.

In certain embodiments, the present invention relates to methods of treatment with a compound above, an analog and/or a pharmaceutically acceptable salt thereof. In certain embodiments, the therapeutic preparation may be enriched to provide predominantly one enantiomer of a compound.

In certain embodiments, the therapeutic preparation may be enriched to provide predominantly one diastereomer of a compound. A diastereomerically enriched mixture may comprise, for example, at least 60 mol percent of one diastereomer, or more preferably at least 75, 90, 95, or even 99 mol percent.

In certain embodiments, the present invention relates to methods of treatment with a compound of Formula I, A, B or C, or a pharmaceutically acceptable salt thereof. In certain embodiments, the therapeutic preparation may be enriched to provide predominantly one enantiomer of a compound.

In certain embodiments, the therapeutic preparation may be enriched to provide predominantly one diastereomer of a compound. In certain embodiments, the present invention provides a pharmaceutical preparation suitable for use in a human patient, comprising any of the compounds shown above, and one or more pharmaceutically acceptable excipients. In certain embodiments, the pharmaceutical preparations may be for use in treating or preventing a condition or disease as described herein, in certain embodiments, the pharmaceutical preparations have a low enough pyrogen activity to be suitable for use in a human patient. Compounds of any of the above structures may be used in the manufacture of medicaments for the treatment of any diseases or conditions disclosed herein.

Various other objects, features and attendant advantages of the present invention will become fully appreciated as the same becomes better understood when considered in conjunction with the accompanying drawings, in which like reference characters designate the same or similar parts throughout the several views, and wherein:

### BRIEF DESCRIPTION OF THE FIGURES

FIG. 1 is a histogram that shows the effects of BCN057 on the survival of pancreatic cancer cells.

FIG. 2A is a group of images showing pancreatic cancer cell growth after treatment with 10 μM BCN057 at time 0, 15, 30, 60, 120 and 240 minutes.

FIG. 2B is a line plot showing luminescence (RLU) of Panc-1 cells treated with serial dilutions of BNC057.

FIG. 2C is a histogram showing the relative expression of qPCR analysis of pro apoptosis and anti-apoptosis markers in Panc-1 cells: Bax, Caspase-3, Akt-1 and BCl-2.

FIG. 3A is a western blot showing GSK3P phosphorylation at S-9 Increases following the addition of 10 μM BCN057 in Panc-1 Cells.

FIG. 3B is a histogram showing the relative expression as a ratio of phosphorylated GSK3b/total GSK3b protein.

FIG. 4A is western blot showing that BCN057 increases JNK dependent phosphorylation in KRAS mutant Panc-1 cancer cells after drug treatment. Phosphorylation of c-Jun at T-91/93 is known to induce apoptosis. Total c-Jun protein level was unchanged.

FIG. 4B is a western blot of PTEN protein expression over 24 hours with βActin stain from the same sample with vehicle or BCN057 treatment (10 uM) at the indicated time points (20 minutes, 1 hour, 6 hours, 12 hours and 24 hours).

FIG. 4C is a histogram of the ratio of PTEN to βActin densitometry showing the increase in PTEN level.

FIG. 5A is a histogram showing the effects of BCN057 and 5-FU on the survival of pancreatic cancer cell lines after incubation of all drugs at 72 hrs. Control (ND) received no drugs. Study groups include 5FU (50 uM), 057 (BCN057 at 2, 5, 10, and 20 μM or 5FU+BCN057 (BCN057 at 2, 5, 10, and 20 μM in combination with 5FU at 50 μM).

FIG. 5B is a histogram showing the relative LGR-5 and BMI-1 expression in Panc-1 cells treated with 10 μM 057.

FIG. 5C is a group of images showing that BCN057 protects GI epithelium and the LGR5 stem cells (green) from 5FU cytotoxicity.

FIG. 5D is a histogram showing quantitation by % of LGR5+ cells remaining in 1 mM sections at the crypt base showing prevention of 5-FU induced apoptosis in LGR5+ stem cells in GI.

FIG. 6 is a histogram showing cytotoxicity (% viability) of Oxaliplatin and Irinotecan, individually or in combination with 5FU and BCN057 on Panc-1 cells.

FIG. 7A is a western blot showing PD-1 expression on human T-lymphocytes co-cultured with Panc-1 cells with no drug (vehicle) or 1, 6 and 12 hours with BCN057.

FIG. 7B is a histogram (concentration v. luminescence) that demonstrates the Inhibition of GSK3P induces survival of Lymphocytes when exposed to radiation.

FIG. 8 is a western blot that shows PDL-1 expression (red arrow) on Panc-1 cells co-cultured with human T-lymphocytes with no drug (vehicle) or 2.5, 5, 7.5 and 10 hrs. with BCN057.

FIG. 9 is a graph of viability versus concentration of BCN077. The study was conducted using the human KRAS G12D mutant colorectal cancer cell line LS174T46 established from a Duke's type B adenocarcinoma BCN077 which shows potent activity.

FIG. 10A shows the structure of BCN076.

FIG. 10A shows the structure of BCN036.

FIG. 10B shows the structure of BCN077.

FIG. 10C shows the structure of BCN078.

FIG. 11 is a graph that shows the inhibition of proliferation of KRAS mutant G12D invasive pancreatic cancer cell lines by analogues of BCN057 076, 077, 036 and 078.

FIG. 12 is a western blot of PD1 expression in co-culture of T-cells and Panc1 cells treated with BCN036 or BCN076 at various time points.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present disclosure (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the disclosure otherwise claimed. No language in the present specification should be construed as indicating any non-claimed element essential to the practice of the disclosure.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

The term “cancer” can include one or more of Adenoid Cystic Carcinoma, Adrenal Gland Cancer, Amyloidosis, Anal Cancer, Ataxia-Telangiectasia, Atypical Mole Syndrome, Basal Cell Carcinoma, Bile Duct Cancer, Birt Hogg Dube Syndrome, Bladder Cancer, Bone Cancer, Brain Tumor, Breast Cancer, Breast Cancer in Men, Carcinoid Tumor, Cervical Cancer, Colorectal Cancer, Ductal Carcinoma, Endometrial Cancer, Esophageal Cancer, Gastric Cancer, Gastrontestinal Stromal Tumor (GIST), HER2-Positive Breast Cancer, Islet Cell Tumor, Juvenile Polyposis Syndrome, Kidney Cancer, Laryngeal Cancer, Leukemia—Acute Lymphoblastic Leukemia, Leukemia—Acute Lymphocytic (ALL), Leukemia—Acute Myeloid AML, Leukemia—Adult, Leukemia—Childhood, Leukemia—Chronic Lymphocytic—CLL, Leukemia—Chronic Myeloid—CML, Liver Cancer, Lobular Carcinoma, Lung Cancer, Lung Cancer—Small Cell, Lymphoma—Hodgkin's, Lymphoma—Non-Hodgkin's, Malignant Glioma, Melanoma, Meningioma, Multiple Myeloma, Myelodysplastic Syndrome (MDS), Nasopharyngeal Cancer, Neuroendocrine Tumor, Oral Cancer, Osteosarcoma, Ovarian Cancer, Pancreatic Cancer, Pancreatic Neuroendocrine Tumors, Parathyroid Cancer, Penile Cancer, Peritoneal Cancer, Peutz-Jeghers Syndrome, Pituitary Gland Tumor, Polycythemia Vera, Prostate Cancer, Renal Cell Carcinoma, Retinoblastoma, Salivary Gland Cancer, Sarcoma, Sarcoma—Kaposi, Skin Cancer, Small Intestine Cancer, Stomach Cancer, Testicular Cancer, Thymoma, Thyroid Cancer, Uterine (Endometrial) Cancer, Vaginal Cancer and Wilms' Tumor. The term “blood cancer” can include one or more of leukemia, lymphoma, myeloma, myelodysplastic syndromes and myeloproliferative neoplasms.

The term “derivative” can refer to any compound having the same or a similar core structure to the compound but having at least one structural difference, including substituting, deleting, and/or adding one or more atoms or functional groups. The term “derivative” does not mean that the derivative is synthesized from the parent compound either as a starting material or intermediate, although this may be the case. The term “derivative” can include prodrugs, or metabolites of the parent compound. Derivatives include compounds in which carboxyl groups in the parent compound have been derivatized to form methyl and ethyl esters, or other types of esters or hydrazides. Derivatives include compounds in which hydroxyl groups in the parent compound have been derivatized to form O-acyl or O-alkyl derivatives. Derivatives include compounds in which a hydrogen bond donating group in the parent compound is replaced with another hydrogen bond donating group such as OH, NH, or SH. Derivatives include replacing a hydrogen bond acceptor group in the parent compound with another hydrogen bond acceptor group such as esters, ethers, ketones, carbonates, tertiary amines, imine, thiones, sulfones, tertiary amides, and sulfides. Derivatives can also include the salt forms, such as pharmaceutically acceptable salt forms of a parent compound or derivative thereof.

The term “fibrosis” refers to a condition with the formation of excess fibrous connective tissue in an organ or tissue in a reparative or reactive process. This can be a reactive, benign, or pathological state.

As used herein, “mitigating” means reducing one or more negative symptoms of a condition, relative to a cell, organ, tissue, or organism displaying the symptom or condition for the same amount of time, but untreated.

In some embodiments, contacting the cell, organ, tissue, or organism the present compounds may comprise administering a therapeutically effective amount of the compound to a subject. As used herein, a “therapeutically effective amount” is an amount sufficient to mitigate the negative symptom or condition.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

The term “pharmaceutically acceptable prodrugs” as used herein represents those prodrugs of the compounds of the present disclosure that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the present disclosure. A discussion is provided in Higuchi et al., “Prodrugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The term “chemotherapy” refers to a type of cancer treatment that uses one or more anti-cancer drugs (i.e., chemotherapeutic agents). Chemotherapy can be given with a curative intent (typically with combinations of drugs), or it can be used to prolong life or to reduce symptoms (i.e. palliative chemotherapy). Conventional chemotherapeutic agents are cytotoxic by means of interfering with cell division (mitosis). Common side effects of chemotherapy include myelosuppression, mucositis (inflammation of the lining of the digestive tract) and alopecia.

The term “KRAS” refers to a gene that acts as an “on/off” switch in cell signaling. When it functions normally, it controls cell proliferation. It can be allosterically activated and recruits and activates proteins necessary for the propagation of growth factors, along with other cell signaling receptors such as c-Raf and PI 3-kinase. Negative signaling can be disrupted when it is mutated causing cells to over-proliferate and develop into cancer.

The term “PD-1,” “Programmed cell death protein 1” or “CD279” refers to a protein on the surface of T and B cells that has a role in regulating the immune system's response to the cells of the human body by down-regulating the immune system and promoting self-tolerance by suppressing T cell inflammatory activity. This prevents autoimmune diseases, but it can also prevent the immune system from killing cancer cells. PD-1 is an immune checkpoint and guards against autoimmunity through two mechanisms. First, it promotes apoptosis (programmed cell death) of antigen-specific T-cells in lymph nodes. Second, it reduces apoptosis in regulatory T cells (anti-inflammatory, suppressive T cells). PD-1 inhibitors, a class of drugs that block PD-1, activate the immune system to attack tumors and are used to treat certain types of cancer. Engagement of PD-1 by either of its ligands, PD-L1 or PD-L2, on an adjacent cell inhibits TCR signaling and TCR-mediated proliferation, transcriptional activation and cytokine production. This prevents autoimmune diseases, but it can also prevent the immune system from killing cancer cells. Therapeutic antibodies designed to block the PD-1/PD-L1 interaction have potential for the treatment of cancer.

Programmed death-ligand 1 (PD-L1) also known as cluster of differentiation 274 (CD274) or B7 homolog 1 (B7-H1) is a protein that in humans is encoded by the CD274 gene. PD-L1 binds to its receptor, PD-1, found on activated T cells, B cells, and myeloid cells, to modulate activation or inhibition.

LAG3 (also known as CD223) is a 503 amino acid transmembrane protein receptor expressed on activated CD4 and CD8T cells, y 5 T cells, natural killer T cells, B cells, natural killer cells, plasmacytoid dendritic cells, and regulatory T cells. LAG3 is an immunoglobulin (Ig) superfamily member. The primary function of LAG3 is to attenuate the immune response. Binding of LAG3 to MHC class II molecules results in delivery of negative signals to LAG3 expressing cells and down-regulation of antigen-dependent CD4 and CD8T cellular responses. LAG3 negatively regulates the ability of T cells to proliferate, produce cytokines, and lyse target cells, referred to as “depletion” of T cells. LAG3 is reported to play a role in enhancing regulatory t (treg) cell function.

T cell costimulatory and costimulatory molecules play an essential role in regulating T cell activation, subpopulation differentiation, effector function and survival. After recognition of the cognate peptide-MHC complex on the antigen presenting cell by the T cell receptor, the signaling co-receptor and the T cell receptor co-localize at the immune synapse where it cooperates with TCR signaling to promote or inhibit T cell activation and function. The function of lymphocyte activation gene-3 (LAG3) is one such “point of immunological testing” in the mediation of peripheral T cell tolerance.

An “immunoregulator” refers to a substance, an agent, a signaling pathway or a component thereof that regulates an immune response. “Regulating,” “modifying” or “modulating” an immune response refers to any alteration in a cell of the immune system or in the activity of such cell. Such regulation includes stimulation or suppression of the immune system which may be manifested by an increase or decrease in the number of various cell types, an increase or decrease in the activity of these cells, or any other changes which can occur within the immune system. Both inhibitory and stimulatory immunoregulators have been identified, some of which may have enhanced function in the cancer, infectious disease or neurodegenerative microenvironment.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication, condition or biochemical indicia associated with a disease.

The term “glycogen synthase kinase 3p” or “GSK3P” refers to an enzyme that in humans is encoded by the GSK3B gene. It is integrally tied to pathways of cell proliferation and apoptosis. GSK-3 β has been shown to phosphorylate Beta-catenin, thus targeting it for degradation. GSK-3 β is therefore a part of the canonical Beta-catenin/Wnt pathway, which signals the cell to divide and proliferate. It also participates in a number of apoptotic signaling pathways by phosphorylating transcription factors that regulate apoptosis. GSK-3β is also over expressed in several types of cancers, like colorectal, ovarian, and prostate cancer. GSK-3β inhibitors also aid in the treatment of Alzheimer's disease, stroke and mood disorders, including bipolar disorder. In diabetes, GSK-3β inhibitors increase insulin sensitivity, glycogen synthesis, and glucose metabolism in skeletal muscles, and reduce obesity by affecting the adipogenesis process.

The term “SFRP” or “secreted frizzled-related protein” refers to a Wnt signaling pathway inhibitor which is part of the soluble frizzled-related proteins (sFRPS). sFRPS function as modulators of Wnt signaling through direct interaction with Wnts. Five mammalian sFRPs have been identified (sFRP-1, sFRP-2, sFRP-3, sFRP-4 and sFRP-5). These proteins consist of approximately 300 amino acids containing a signal sequence, a Frizzled-like cysteine-rich domain (CRD), and a small hydrophilic C-terminal domain. As a group, sFRPs are expressed in a variety of embryonic and adult tissues, suggesting a common mechanism for inhibiting Wnt signaling. Individual family members, however, have specific spatial and temporal expression patterns.

The term “Wnt signaling pathways” refers to a group of signal transduction pathways which begin with proteins that pass signals into a cell through cell surface receptors. The canonical Wnt pathway leads to regulation of gene transcription.

The term “RIGS” or “radiation-induced gastrointestinal syndrome” results from a combination of direct cytocidal effects of irradiation on intestinal crypt cells and stromal cells with loss of the mucosal barrier and symptoms ranging from diarrhea, electrolyte imbalance, weight loss and death. A significant proportion of patients experience radiation-induced toxicity due to damage to normal tissue in the irradiation field. The use of chemical or biological approaches aimed at reducing or preventing normal tissue toxicity induced by radiotherapy is a long-held goal.

The term “radiation therapy” or “radio therapy” refers to a therapy using ionizing radiation, generally as part of cancer treatment to control or kill malignant cells. Radiation therapy is commonly applied to the cancerous tumor because of its ability to control cell growth. Ionizing radiation works by damaging the DNA of cancerous tissue leading to cellular death. The main side effects are fatigue and skin irritation. Acute side effects can include nausea and vomiting, damage to the epithelial surfaces, mouth, throat and stomach sores, swelling, intestinal discomfort. RIGS (radiation induced gastrointestinal syndrome) is a common term used to describe side effect related to the gastrointestinal system.

The term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease. Treatment can be prophylactic and/or therapeutic. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

The term “unit dosage form” or “unit” as used herein refers to physically discrete units suitable as unitary dosages for human and animal subjects, each unit containing a predetermined quantity of the compound calculated in an amount sufficient to produce the desired effect in association with a pharmaceutically acceptable, diluent, carrier or vehicle. The specifications for the novel unit dosage forms of the present disclosure depend on the particular compound employed and the effect to be achieved, and the pharmacodynamics associated with each compound in the subject.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Geometric isomers can also exist in the compounds of the present disclosure. The present disclosure encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the E and Z isomers.

Substituents around a carbon-carbon double bond alternatively can be referred to as “cis” or “trans,” where “cis” represents substituents on the same side of the double bond and “trans” represents substituents on opposite sides of the double bond. The arrangements of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The compounds disclosed herein may exist as tautomers and both tautomeric forms are intended to be encompassed by the scope of the present disclosure, even though only one tautomeric structure is depicted.

The term “alkoxy” represents a chemical substituent of formula —OR, where R is an optionally substituted C₁-C₆ alkyl group, unless otherwise specified. In some embodiments, the alkyl group can be substituted, e.g., the alkoxy group can have 1, 2, 3, 4, 5 or 6 substituent groups as defined herein.

The term “alkoxyalkyl” represents a heteroalkyl group, as defined herein, that is described as an alkyl group that is substituted with an alkoxy group. Exemplary unsubstituted alkoxyalkyl groups include between 2 to 12 carbons. In some embodiments, the alkyl and the alkoxy each can be further substituted with 1, 2, 3, or 4 substituent groups as defined herein for the respective group.

The terms “alkyl,” “alkenyl” and “alkynyl” include straight-chain, branched-chain and cyclic monovalent substituents, as well as combinations of these, containing only C and H when unsubstituted. Examples include methyl, ethyl, isobutyl, cyclohexyl, cyclopentylethyl, 2-propenyl, 3-butynyl, and the like. The term “cycloalkyl,” as used herein, represents a monovalent saturated or unsaturated non-aromatic cyclic alkyl group having between three to nine carbons (e.g., a C3-C9 cycloalkyl), unless otherwise specified, and is exemplified by cyclopropyl, cyclobutyl, cyclopentyl, cyclohexyl, cycloheptyl, bicyclo[2.2.1.]heptyl, and the like. When the cycloalkyl group includes one carbon-carbon double bond, the cycloalkyl group can be referred to as a “cycloalkenyl” group. Exemplary cycloalkenyl groups include cyclopentenyl, cyclohexenyl, and the like. Typically, the alkyl, alkenyl and alkynyl groups contain 1-12 carbons (e.g., C₁-C₁₂ alkyl) or 2-12 carbons (e.g., C₂-C₁₂ alkenyl or C₂-C₁₂ alkynyl). In some embodiments, the alkyl groups are C₁-C₈, C_{I}-C₆, C_{I}-C₄, C₁-C₃, or C₁-C₂ alkyl groups; or C₂-C₈, C₂-C₆, C₂-C₄, or C₂-C₃ alkenyl or alkynyl groups. Further, any hydrogen atom on one of these groups can be replaced with a substituent as described herein.

Heteroalkyl, heteroalkenyl and heteroalkynyl are similarly defined and contain at least one carbon atom but also contain one or more 0, S or N heteroatoms or combinations thereof within the backbone residue whereby each heteroatom in the heteroalkyl, heteroalkenyl or heteroalkynyl group replaces one carbon atom of the alkyl, alkenyl or alkynyl group to which the heteroform corresponds. In some embodiments, the heteroalkyl, heteroalkenyl and heteroalkynyl groups have C at each terminus to which the group is attached to other groups, and the heteroatom(s) present are not located at a terminal position. As is understood in the art, these heteroforms do not contain more than three contiguous heteroatoms. In some embodiments, the heteroatom is O or N. The term “heterocyclyl,” as used herein represents cyclic heteroalkyl or heteroalkenyl that is, e.g., a 3-, 4-, 5-, 6- or 7-membered ring, unless otherwise specified, containing one, two, three, or four heteroatoms independently selected from the group consisting of nitrogen, oxygen, and sulfur. The 5-membered ring has zero to two double bonds, and the 6- and 7-membered rings have zero to three double bonds. The term “heterocyclyl” also represents a heterocyclic compound having a bridged multicyclic structure in which one or more carbons and/or heteroatoms bridges two non-adjacent members of a monocyclic ring, e.g., a quinuclidinyl group. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopenlane ring, a cyclopentene ring, or another monocyclic heterocyclic ring, such as indolyl, quinolyl, isoquinolyl, tetrahydroquinolyl, benzofuryl, benzothienyl and the like.

The term “alkylsulfonyl,” as used herein, represents a heteroalkyl group that is described as an optionally substituted alkyl group, as described herein, that includes an —S(O)₂— group.

The term “amino,” as used herein, represents —N(R)₂, wherein each R is, independently, H, OH, NO₂, N(R)₂, SO₂OR, SO₂R, SOR, SO₂N(R)₂, SON(R)₂, an N-protecting group, alkyl, alkenyl, alkynyl, alkoxy, aryl, alkaryl, cycloalkyl, alkcycloalkyl, heterocyclyl (e.g., heteroaryl), alkheterocyclyl (e.g., alkheteroaryl), or two R combine to form a heterocyclyl or an N-protecting group, and wherein each R^{N2} is, independently, H, alkyl, or aryl. In a preferred embodiment, amino is —NH₂, or —NHR, wherein R is, independently, OH, NO₂, NH₂, NR₂, SO₂OR, SO₂R, SOR, SO₂N(R)₂, SON(R)₂, alkyl, or aryl, and each R can be H, alkyl, or aryl. The term “aminoalkyl,” as used herein, represents a heteroalkyl group, as defined herein, that is described as an alkyl group, as defined herein, substituted by an amino group, as defined herein. The alkyl and amino each can be further substituted with 1, 2, 3, or 4 substituent groups as described herein for the respective group. For example, the alkyl moiety may comprise an oxo (═O) substituent.

“Aromatic” moiety or “aryl” moiety refers to any monocyclic or fused ring bicyclic system which has the characteristics of aromaticity in terms of electron distribution throughout the ring system and includes a monocyclic or fused bicyclic moiety such as phenyl or naphthyl; “heteroaromatic” or “heteroaryl” also refers to such monocyclic or fused bicyclic ring systems containing one or more heteroatoms selected from O, S and N. The inclusion of a heteroatom permits inclusion of 5-membered rings to be considered aromatic as well as 6-membered rings. Thus, typical aromatic/heteroaromatic systems include pyridyl, pyrimidyl, indolyl, benzimidazolyl, benzotriazolyl, isoquinolyl, quinolyl, benzothiazolyl, benzofuranyl, thienyl, fury!, pyrrolyl, thiazolyl, oxazolyl, isoxazolyl, benzoxazolyl, benzoisoxazolyl, imidazolyl and the like. Because tautomers are theoretically possible, phthalimido is also considered aromatic. Typically, the ring systems contain 5-12 ring member atoms or 6-10 ring member atoms. In some embodiments, the aromatic or heteroaromatic moiety is a 6-membered aromatic rings system optionally containing 1-2 nitrogen atoms. More particularly, the moiety is an optionally substituted phenyl, pyridyl, indolyl, pyrimidyl, pyridazinyl, benzothiazolyl or benzimidazolyl, pyrazolyl, imidazolyl, isoxazolyl, thiazolyl, benzothiazolyl, indolyl. Even more particularly, such moiety is phenyl, pyridyl, or pyrimidyl and even more particularly, it is phenyl. “O-aryl” or “O-heteroaryl” refers to aromatic or heteroaromatic systems which are coupled to another residue through an oxygen atom. A typical example of an O-aryl is phenoxy. Similarly, “arylalkyl” refers to aromatic and heteroaromatic systems which are coupled to another residue through a carbon chain, saturated or unsaturated, typically of C₁-C₂, C₁-C₆, or more particularly C₁-C₄ or C₁-C₃ when saturated or C₂-C₈, C₂-C₆, C₂-C₄, or C₂-C₃ when unsaturated, including the heteroforms thereof. For greater certainty, arylalkyl thus includes an aryl or heteroaryl group as defined above connected to an alkyl, heteroalkyl, alkenyl, heteroalkenyl, alkynyl or heteroalkynyl moiety also as defined above. Typical arylalkyls would be an aryl(C₆-C₁₂)alkyl(C₁-C₈), aryl(C₆-C₁₂)alkenyl(C₂-C₈), or aryl(C₆-C₁₂)alkynyl(C₂-C₈), plus the heteroforms. A typical example is phenylmethyl, commonly referred to as benzyl.

Halo may be any halogen atom, especially F, Cl, Br, or I, and more particularly it is fluoro or chloro.

The term “haloalkyl,” as used herein, represents an alkyl group, as defined herein, substituted by a halogen group (i.e., F, Cl, Br, or I). A haloalkyl may be substituted with one, two, three, or, in the case of alkyl groups of two carbons or more, four halogens. Haloalkyl groups include perfluoroalkyls. In some embodiments, the haloalkyl group can be further substituted with 1, 2, 3, or 4 substituent groups as described herein for alkyl groups.

The term “hydroxy,” as used herein, represents an —OH group.

The term “hydroxyalkyl,” as used herein, represents an alkyl group, as defined herein, substituted by one to three hydroxy groups, with the proviso that no more than one hydroxy group may be attached to a single carbon atom of the alkyl group, and is exemplified by hydroxymethyl, dihydroxypropyl, and the like.

In general, a substituent group (e.g., alkyl, alkenyl, alkynyl, or aryl (including all heteroforms defined above) may itself optionally be substituted by additional substituents. The nature of these substituents is similar to those recited with regard to the substituents on the basic structures above. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments, and is not included. For example, where a group is substituted, the group may be substituted with 1, 2, 3, 4, 5, or 6 substituents. Optional substituents include, but are not limited to: C₁-C₆ alkyl or heteroaryl, C₂-C₆ alkenyl or heteroalkenyl, C₂-C₆ alkynyl or heteroalkynyl, halogen; aryl, heteroaryl, azido (—N₃), nitro (—NO₂), cyano (—CN), acyloxy (—OC(═O)R′), acyl (—C(═O)R′), alkoxy (—OR′), amido (—NR′C(═O)R″ or —C(═O)NR′R″), amino (—NR′R″), carboxylic acid (—CO₂H), carboxylic ester (—CO₂R′), carbamoyl (—OC(═O)NR′R″ or —NRC(═O)OR′), hydroxy (—OH), isocyano (—NC), sulfonate (—S(═OHOR), sulfonamide (—S(═OHNRR′ or —NRS(═O)₂R′), or sulfonyl (—S(═O)₂R), where each R or R′ is selected, independently, from H, CI-C6 alkyl or heteroaryl, C₂-C₆ alkenyl or heteroalkenyl, C₂-C₆ alkynyl or heteroalkynyl, aryl, or heteroaryl. A substituted group may have, for example, 1, 2, 3, 4, 5, 6, 7, 8, or 9 substituents.

Typical optional substituents include independently halo, CN, NO₂, CF₃, OCF₃, COOR, CONR^{Y}₂, OR, SR, SOR, SO₂R, NR₂, NR(CO)R, NRC(O)OR, NRC(O)NR₂, NRSO₂NR₂, or NRSO₂R, wherein each R is independently H or an optionally substituted group selected from alkyl, alkenyl, alkynyl, heteroalkyl, heteroalkenyl, heteroalkynyl, heteroaryl, and aryl (all as defined above); or the substituent may be an optionally substituted group selected from alkyl, alkenyl, alkynyl, heteroalkyl, heteroalkenyl, heteroalkynyl, aryl, heteroaryl, O-aryl, O-heteroaryl and arylalkyl.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

### BCN057 and Analogs

BCN057, also known as YEL002, is represented by the following structure:

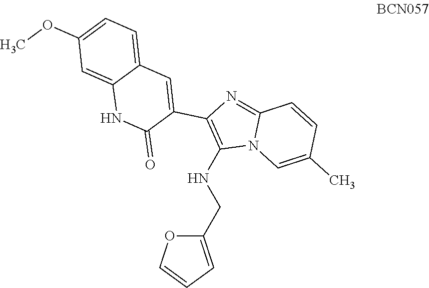

It will be appreciated that the invention covers compounds of BCN057, analogs and salts thereof. In one embodiment, the invention relates to compounds of BCN057 in the form of a free base. In another embodiment, the invention relates to compounds of BCN057 or a pharmaceutically acceptable salt thereof.

Because of their potential use in medicine, salts of the compound of BCN057 may be preferred as pharmaceutically acceptable salts. Suitable pharmaceutically acceptable salts can include acid addition salts. A pharmaceutically acceptable salt can be readily prepared by using a desired acid or base as appropriate. The resultant salt can precipitate from solution and be collected by filtration or recovered by evaporation of the solvent. The compound can exist as a stereoisomer, tautomer, pharmaceutical acceptable salt, or hydrate thereof. Some analogs of BCN057 are presented below.

Embodiments include methods of treatment that include administering a therapeutically effective amount of a compound having the structure of Formula B:

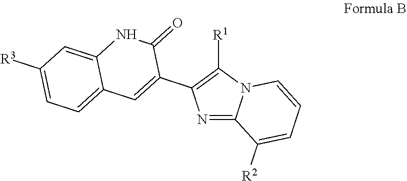

wherein:
R¹ is an alkyl, alkyl amine, or an ether;
R² is H or CH₃; and
R³ is H or OH.

In some embodiments, the compound is an analog selected from Formula II-XIX, including Compound H, Compound K, Compound BNB-1, Compound BNB-2, Compound BNB-3, Compound BNB-4 and Compound BNB-5 as shown in Table 2, infra.

One embodiment is a compound of Formula I or an analog thereof, as detailed supra.

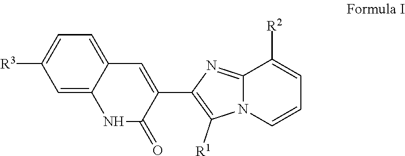

BCN057, along with the compounds and analogs disclosed herein can be prepared according to established methodology in the art of organic synthesis. General methods of synthesizing the compound can be found in, e.g., Stuart Warren and Paul Wyatt, Workbook for Organic Synthesis: The Disconnection Approach, second Edition, Wiley, 2010. Exemplary methods of making the compound is provided in U.S. patent application Ser. No. 13/813,923 and U.S. patent application Ser. No. 14/889,719, herein incorporated by reference. The compounds also include pharmaceutically acceptable salts thereof, prodrugs thereof, hydrates thereof, solvates thereof and polymorphic crystals thereof. The compounds can be administered as pharmaceutical compositions.

### Utility and Administration

BCN057 AND BCN077 and analogs described herein are useful in the methods of the invention and, while not bound by theory, are believed to exert their desirable effects in part through their ability to modulate Wnt-β catenin signaling. The Wnt pathway is involved in tissue development in embryos and tissue maintenance in adults. It controls a specific set of genes that that control cell growth, movement and cell survival. Chronic activation of these genes and aberrant activation of the Wnt pathway leads to uncontrolled cell growth and survival and can consequently drive cancer formation in a range of tissues including colon, skin, liver and ovary.

The Wnt signaling pathways are a group of signal transduction pathways which begin with proteins that pass signals into a cell through cell surface receptors. Aberrant activation of the Wnt pathway is implicated in human cancers, particularly those of the gastrointestinal (GI) tract. Inhibition of aberrant Wnt pathway activity in cancer cell lines can block their growth, presenting the possibility of new therapeutics.

Wnt ligands bind to LRP5/6 and Frizzled co-receptors present on epithelial crypt cells, leading to β-catenin stabilization and nuclear translocation where it binds to the nuclear transcription factor TCF4 to drive a gene-expression program that supports stem cell maintenance, proliferation and differentiation. Activation of Wnt/β-catenin signaling is also crucial for crypt regeneration following injury. Studies have demonstrated that Respondin 1 (RSPO1), an ISC growth factor and LGR5 receptor agonist, activates Wnt/βcatenin pathway to repair and regenerate the intestine following chemo-radiation-induced injury. DKK1, a negative regulator Wnt/β-catenin pathway, impairs the RSPO1-induced intestinal regeneration.

In the absence of a Wnt signal, the transcriptional activator β-catenin is actively degraded in the cell. Phosphorylated β-catenin is subsequently recognized and ubiquitinylated, resulting in its proteasomal degradation. Levels of free β-catenin consequently remain low, which allows the DNA-binding T-cell factor/lymphoid enhancer factor (Tcf/Lef) proteins to interact with transcriptional co-repressors to block target gene expression in the nucleus.

In addition to treating cancer, therapies that control Wnt/β-catenin signaling also potential in treating other ailments, including fibrosis, inflammatory conditions, bone growth, wound healing, osteoporosis, alopecia (i.e. hair loss), depression and viral infection. Accordingly, there is a need for compounds and methods that can inhibit and/or modulate Wnt/β-catenin signaling.

### BCN057 and Analogs—Cancer Treatment and Therapies

Studies have demonstrated that the Wnt pathway is permanently activated in both inherited familial adenomatous polyposis and spontaneous forms of colon cancer. Chronic activation of the Wnt pathway in these cells drives their expansion into benign adenomas (also termed polyps), which frequently progress to invasive colon carcinoma. Approximately 90% of sporadic colon cancers show aberrant Wnt signaling activity, usually as the result of mutations in APC.

BCN057 and BCN077 are anti-neoplastic small molecules that are effective in treating cancer, particularly pancreatic and gastrointestinal (GI) cancers. It can function with dual actions. It induces apoptosis in cancer cells. Second, it can promote growth and survival of epithelial cells. BCN057 and BCN077 are particularly effective against pancreatic and gastrointestinal (GI) cancers. BCN057 and BCN077 have demonstrated antineoplastic effects against “KRAS” cancers.

Use in Conjunction with Radio/Chemo Therapies

BCN057 and BCN077 are effective in treating cancer, particularly pancreatic and gastrointestinal (GI) cancers. In one aspect, disclosed herein, is a method of treating radiation induced gastrointestinal syndrome (RIGS) in a subject in need thereof, the method comprising the step of administering to the subject a therapeutically effective amount of a compound of BCN057, BCN077 or an analog thereof. The analog can be one or more of the compounds of Formula II-XIX. BCN057 (and BCN077) can mitigate RIGS and improve the therapeutic ratio for abdominal radiotherapy. Further, BCN057 and BCN077 mitigate radiation induced mucositis, including oral mucositis, GI mucositis, e.g., of the throat, stomach and intestines, enteritis and proctitis. Further, it can prevent and treat damage to epithelial tissue from radiation and chemotherapy. The compounds are also useful for treating or preventing these radiation syndromes associated with radiation therapy.

### Combination Therapies

Combination therapy can be particularly effective with drugs that work by different mechanisms, thereby decreasing the likelihood that resistant cancer cells will develop. When drugs with different effects are combined, each drug can be used at its optimal dose, without intolerable side effects. BCN057 (and BCN077) and a second medicament can be combined for therapeutic benefit. The second medicant can have a different mechanism. Alternatively, it can use the same mechanism as BCN057 for therapeutic benefit. The combination can act via a synergistic effect. Combination therapies can also include additional (e.g., a third, fourth, fifth, etc.) medicaments. BCN512 can be used in combination therapies in the same manner.

### Treatment of Viral Infections

Additional embodiments include the use of BCN057, BCN077 and analogs of thereof for treatment of viral infections. A viral disease infection occurs when the body is invaded by pathogenic viruses, and infectious virus particles attach to and enter susceptible cells. The host immune response can mediate disease and excessive inflammation. The stimulation of the innate and adaptive immune system in response to viral infections destroys infected cells, which can lead to severe pathological consequences to the host (i.e. virus-induced immunopathology). Specifically, immunopathology is caused by the excessive release of antibodies, interferons and pro-inflammatory cytokines, activation of the complement system, or hyperactivity of cytotoxic T cells. Secretion of interferons and other cytokines can trigger cell damage, fever and flu-like symptoms. In severe cases of certain viral infections, as in avian H5N1 influenza in 2005, aberrant induction of the host immune response can elicit a flaring release of cytokines known as a cytokine storm.

Wnt signaling is important for the innate immune response to viruses. Pathogenic viruses suppress b-catenin downstream expression of critical genes to evade the first line of defense in the immune system. B-catenin is essential to the expression of IFN-α/β and the subsequent transcriptional activation of interferon-stimulated genes. IFN-β induces in an auto- and paracrine expression of antiviral-acting genes. Thus BCN057, BCN077 and analogs can prevent pathological consequences such as virus-induced immunopathology.

### Treatment of Depression

Additional embodiments include the use of BCN057, BCN077 and analogs for treatment of depression. Wnt signaling is also important for treating depression. Since the 1950s Lithium carbonate and other salts have been used similarly to treat depression. Lithium works by inhibiting GSK3b in the wnt downstream signaling pathway and transcribing the b-catenin downstream genes.

Recent studies have demonstrated that altered Wnt signalling can play a role in the pathophysiology of mood disorders. β-catenin levels are reduced in the hippocampal CA3 and CA4 regions, and Wnt1 levels are increased in the hippocampal CA4 region of post-mortem schizophrenic brains. Because lithium inhibits glycogen synthase kinase3β (GSK3β), a component of the canonical Wnt pathway, the pathway has been proposed as a specific target in the treatment of bipolar disorders. Thus, BCN057, BCN077 and analogs can be used to treat depression and mood disorders.

### PD-1 Modulation and Treatment of Ailments Related to PD-1 Activity

PD-1 is a receptor for PD-L1 and PD-L2. The PD-1/PD-L signaling pathway plays a pivotal role in peripheral immune tolerance, which prevents inappropriate immune responses under physiological conditions. PD-1 is an immune checkpoint inhibitor demonstrated to reduce the immune system response in cancer and chronic infection. However, in infection or cancer, the alternative role of this pathway leading to immune suppression could cause serious issues.

Studies have demonstrated that PD-1 is involved in various ailments including cancer, autoimmune diseases, chronic Infection and sepsis. Such studies have highlighted the dual role of PD-1 in immune tolerance, and the loss of PD-1 causes autoimmune diseases. Depletion of PD-1+ T cells has given beneficial effect in autoimmune disease, slowing down the inflammation and disease progression. A decrease of PD-1 is predisposed to autoimmunity, as described in experiments of PD-1 blocking or knockout in mice. PD-1 could also be a target of immunotherapies in multiple sclerosis (MS).

The role of PD-1 in cancer has been studied extensively. PD-L1 and/or PD-L2 expression either in tumor or in infiltrating immune cells has been verified in numerous tumors, indicating a role for the PD-1/PD-L1 axis as a prognostic trait and therapeutic target. Autoimmune diseases are defined as aberrant immune responses of an organism to its own cells and tissues. The incidence of autoimmunity is increasing worldwide, PD-1 and PD-Ls have been demonstrated to be involve in the modulation of both central and peripheral tolerance. In particular, during thymocyte development, PD-1 plays a critical role by regulating signaling thresholds during positive selection. Consequently, the population of CD4+CD8+ thymocytes is increased in the absence of PD-1 or PD-1. Moreover, the PD-1 pathway serves as a negative regulator in autoreactive T and B cells to maintain tolerance.

BCN057, BCN077 and analogs described herein are useful in the methods of the invention and, while not bound by theory, are believed to exert their effects in part through their ability to modulate PD-1 activity. Specifically, Applicants propose the use of BCN057 and analogs of BCN057 therapeutically for modulating PD-1 activity. Accordingly, BCN057 can treat particular ailments in which PD-1 is involved. The mechanism involves GSK3P inhibition.

Specifically, Applicants propose that BCN057 and BCN077 inhibit GSK3P and thereby induces a pro-apoptotic phosphorylation pattern on c-Jun in KRAS G12D mutant pancreatic cancer cells leading to the restoration of PTEN expression and consequent apoptosis. Concurrent with its GSK3P inhibition, BCN057 (and BCN077) is a small molecule inhibitor of PD-1 expression on human T-lymphocytes co cultured with human pancreatic cancer cells. BCN057 and BCN077 can promote synthetic lethality specifically to malignant cells and therefore should be considered to improve the therapeutic ratio in pancreatic and epithelial cancer treatment in conjunction with chemotherapy and radiation.

Chronic infection is characterized by persistently high levels of antigen exposure, causing T cells to progressively lose effector functions and progress to exhaustion, similar to the state in cancer. Given its success in the treatment of cancer, anti-PD-1/PD-L1 therapy has potential in the treatment of severe chronic infections. Further, recent studies have demonstrated that PD-1 regulates several metabolic changes in the development of T-cell exhaustion.

Sepsis, associated with high mortality in intensive care units, is characterized by severe immunosuppression after the first proinflammatory hours. Postmortem examination of deceased septic patients highlights key immunological defects that impair host immunity. Sepsis has immunosuppressive mechanisms similar to those of cancer. Given the potency of CTLA-4- and PD-1-specific antibodies in improving host immunity and increasing survival in cancer patients, these agents are expected to open a promising avenue to the development of novel medicines for sepsis

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, pharmaceutical compositions, or methods and uses disclosed herein.

### Materials and Methods

Human Peripheral Blood Pan-T Cells was purchased from Stem Cell Technologies (Vancouver, BC, Canada) and Panc-1 cells were obtained from the Kansas University Medical Center. 5-Fu, Irinotecan, and Oxaloplatin were purchased from Sigma (St. Louis, MO, USA). ImmunoCult-XF T Cell Exp Medium, ImmunoCult HuCD3/CD28/CD2 TCellAct, Hu Recom IL-2 (CHO Expr.) were purchased from Stem Cell Technologies (Vancouver, BC, Canada). The ATPLite Luminescence Assay System and 96-well white, opaque sterile tissue culture treated microplates were purchased from Perkin Elmer (Waltham, Massachusetts, USA). The RealTime Glo Annxin V Apoptosis and Annexin Assay was purchased from Promega (San Luis Obispo, CA, USA). Phospho-GSK-3β (Ser9), PD-L1 (E1L3N). PD-1 (D4W2J), β-Actin, Anti-rabbit IgG, HRP-linked Antibody, Phospho-Akt (Thr308), Phospho-c-Jun (T-93) were purchased from Cell Signaling Technology (Danvers, Massachusetts, USA). 0.4 μm TC Plate Insert with a PC Membrane 6 well plate (thinserts) were purchased from VWR (Radnor Pennsylvania, USA). PD-L1, B7-H1) Monoclonal Antibody (MIH1) conjugated with APC and (PD-1) Monoclonal Antibody (MIH4), conjugated with FITC were purchased from Thermo Fisher Scientific (Waltham, Massachusetts, USA).

Preparation of Compounds: Stock solutions of the following compounds were prepared. 150 mM Oxaloplatin in DMSO, 100 mM Irinotecan in DMSO, 50 mM 5Fu in DMSO, and BCN 057 16 mg/mL BCN057 in 30% Captisol® (β-cyclodextrin sulfobutyl ether sodium).

Cell lines and cell culture: Panc-1 cells were cultured in DMEM medium supplemented with 10% Fetal Bovine Serum and maintained at 37° C.5% CO₂. Human Peripheral Blood Pan-T Cells were cultured in serum free T cells expansion media with 10 ng/ml of rhIL-2, T-cell activator and expanded as per the manufacturer instructions. Panc-1 cells and T-Cells were grown and expanded separately in T75 flasks. For co-culture experiments with Panc-1 cell, 2.5 million T-cells per well were plated in the well using a 6 well plate. 2.5 million Panc-1 Cells were plated in the thinsert which was placed into the well of the 6 well plate in order to have the cells physically separated but share media. The cells were incubated for 24 hours at 37° C., 5% CO₂. At 24 hours the media was replaced with media containing vehicle, or 10 uM BCN057 and were incubated for the following timepoints: 1 hr, 6 hr, 12 hr, 24 hrs. The Panc-1 cells were removed by gently scraping the cells from the thinsert.

Flow Cytometry: Flow cytometry analysis PANC-1 and T-Cells were harvested independently and washed with 1×PBS. The cells were washed with 3×PBS and incubated with antibody 1 hour at room temperature. The cells were then washed with 3×PBS and resuspended with 1×PBS and analyzed by flow cytometry using a NovoCyte flow cytometer (Agilent Technologies Inc. Santa Clara, CA) and the data was analyzed using the NovoExpress software.

Western Blot: Cells were harvested and lysed in cell lysis buffer on ice for 15 minutes and the lysate was centrifuged at 13,000 rpm at 4° C. for 10 minutes. The supernatant was transferred into a new tube for BCA protein quantification assay (Thermo Fisher Scientific) and the samples were normalized. 3× loading buffer (Cell Signaling) was added the protein sample and boiled for 5 minutes. The samples were used for SDS-PAGE analysis and transferred to nitrocellulose membrane. The membrane was blocked with 5% BSA at room temperature for 1 hour and incubated with primary antibody at 4° C. overnight. The membrane was washed with 1×TBST 5 minutes for 3 times and incubated with horseradish peroxidase-conjugated secondary antibody for 1 hour at room temperature. The protein was visualized by SuperSignal West Pico Stable Peroxide Solution (Thermo Fisher Scientific). The blot was visualized by chemiluminescence using a BioRad Versadoc Imaging System.

Viability Assay. To determine the effects of BCN 057 in comparison and combination with irinotecan, Oxaloplatin and 5Fu, the ATPLite Luminescence Assay System was used according to manufacturer's instructions. On day 1, 3,000 Panc-1 cells per well were seeded in 96-well white, opaque sterile tissue culture treated microplates and incubated for 24 hours in complete medium at 37° C., 5% CO₂. On day 2, the media was exchanged with media containing various combinations of 10 uM BCN 057, 25 μM 5-Fu, 75 μM Oxaloplatin, and 50 μM Irinotecan and incubated at 37° C., 5% CO₂. On day 3,4, and 5, the ATP Lite Assay was performed according to manufacturer's instructions for the 24, 48, and 72 hr timepoints. Unless otherwise stated, values were represented as a percent of the control which is the vehicle for each drug. No difference was found in viability between any control vehicle.

QPCR: To compare the mRNA levels of BMI1 and LGR5 target genes in Panc-1 cells treated with BCN057 or vehicle. Quantitative PCR (qPCR) was used to measure the expression of the stem cell markers LGR5, and BMI1 which was detected by real-time PCR using the primer pairs listed in Table 1. Total RNA was extracted using TRIzol kit (Invitrogen, CA, USA)

| Primer | | |
|---|---|---|
| Name | Forward Sequence | Reverse Sequence |
| h-LGR-5 | 5′TCAGTCAGCTGCTCCCGAAT3′ | 5′CGTTTCCCGCAAGACGTAAC3′ |
| h-BMI-1 | 5′ATGCATCGAACAACGAGAATCAA | 5′TCAACCAGAAGTTGCTGATGA |
| | GATCACT3′ | CCC3′ |
| H-Actin-b | 5′TGGATCAGCAAGCAGGAGTATG3′ | 5′GCATTTGCGGTGGACGAT3′ |

Primers used for qPCR of LGR5 and BMI1 in pancreatic cancer cells Panc-1 treated with 10 μM of BCN057 or vehicle.

Statistics. The results are shown by calculating the averages (±Standard deviation) in comparison to untreated controls with the indicated (n). The Percent Viability was calculated by dividing the average of each condition by the average of the untreated control (vehicle). Statistical analysis was performed in excel.

### Example 1

### BCN057 Inhibits Pancreatic Cancer Cell Growth with Restoration of Apoptosis

The first example entailed studying the effect of BCN057 in pancreatic cancer cells. Panc-1 cells, a G12D KRAS mutant cell line was treated with BCN057 for 48 hours. ATPlite™ assay (PerkinElmer) demonstrated a significant decrease in Panc1 cells viability with increasing doses of BCN057.

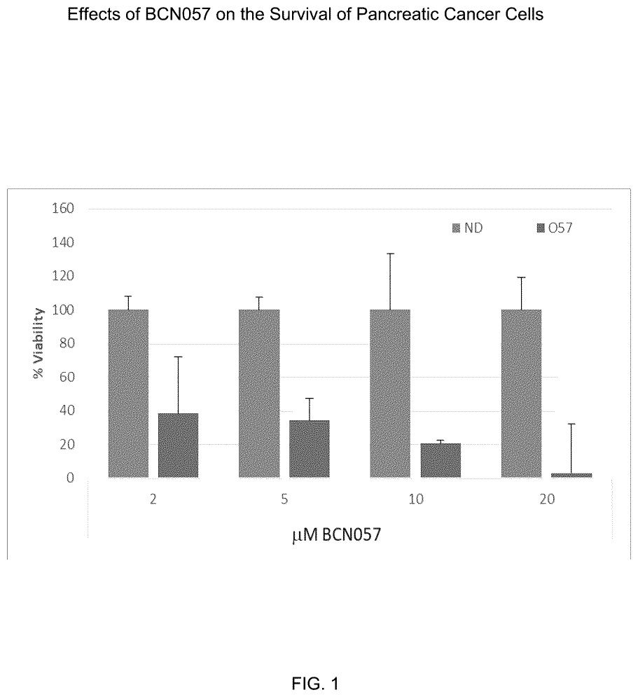

FIG. 1 is a histogram that shows the effects of BCN057 on the survival of pancreatic cancer cell lines as assessed by ATP Lite™ assay (Perkin Elmer) with incubation 72 hours post application of drug. All data points are n=5. Control (ND) received no drug and BCN057 at 2, 5, 10, and 20 μM final concentration. The Percent viability was calculated by dividing the average value for each condition by the average value of the control cells (V) at each time point.

$$Y = Min + \frac{Max - Min}{1 + {( X / {EC}_{50} )}^{Hill coefficient}} \qquad Equation 1$$

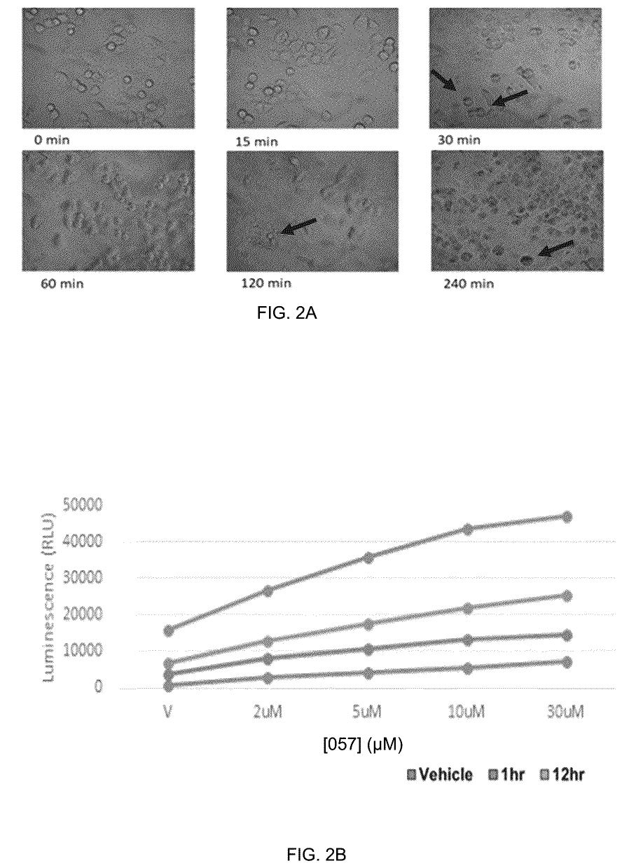

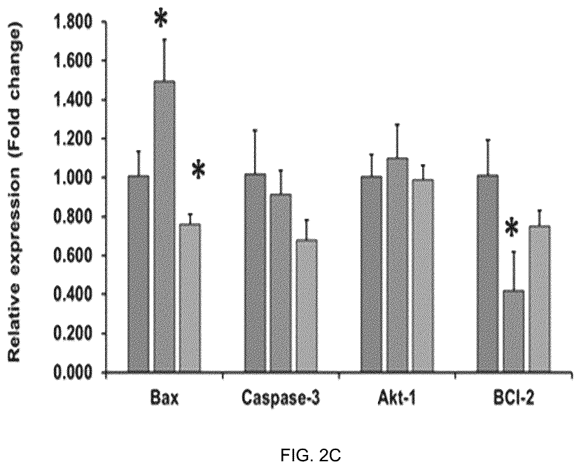

This data was plotted with Quest Graph™ 1050 Calculator using Equation 1 which yielded an IC₅₀ of 1.33 mm. This value compares with other analysis of cytotoxic drugs, such as gemcitabine, on pancreatic ductal adenocarcinoma cells (AsPC-1, BxPC-3, MIA PaCa-2 and Panc-1), where IC₅₀'s ranges from 494 nM to 23.9 μM15. The drug was found to show potent cytotoxicity toward the cells at all concentrations in the dose response curve. Further, these doses span the concentrations found in plasma during efficacy studies in rodents. Subsequently, microscope images appeared to show cell blebbing and fragmentation shortly after application of BCN057 indicating possible apoptosis. FIG. 2A shows a time lapse of pancreatic cancer cell growth after treatment with 10 μM BCN057. Blebbing and cell fragmentation was noted within 20 min of incubation with drug (as shown with the arrows). Cell fragmentation was seen between 20-30 minutes after exposure to the drug.

Because Panc-1 cells possess a heterozygous missense mutation in codon 12 (p.G12D; GGT>GAT of K-RAS) similar to other KRAS mutant tumor cells, the phenotype is a loss in the capacity for apoptosis and consequent chemo resistance. BCN057 treatment appeared to exhibit a rapid and dose dependent restoration of apoptosis as soon as 20 minutes of exposure to the drug.

To validate the involvement of apoptosis in BCN057 induced cell death, the Panc1 cells with/without BCN057 were subjected to Anexin V staining. Anexin V staining was performed up to 6 hours and measured as a function of dose response of BCN057. Dose and time dependent increase in apoptosis was detected in Panc1 cells treated with BCN057. FIG. 2B is a graph of Annexin V Apoptosis and Necrosis Assay in Panc-1 cells. Panc-1 cells were exposed to serial dilutions of BCN057 in the presence or absence of the RealTime-Glo™ Annexin V Apoptosis and Necrosis Assay Reagent. The plate was incubated at 37° C./5% CO₂ and luminescence (Annexin V binding) was measured over a period of six hours. No change was observed in fluorescence over the same time period. Apoptosis appeared to increase with increasing concentrations of BCN057. Apoptosis was observed in a dose dependent fashion and time dependent with time points at 10 minutes, 30 minutes, 1 hour and 6 hours.

Further qPCR analysis was performed on Bax, BCl-2, Akt-1 and Caspase-3 which demonstrated that BCN057 induces expression of the pro-apoptotic gene Bax but inhibited anti-apoptotic Bcl2 gene expression in PANC1 cells at 1-hour post treatment. FIG. 2C is a histogram of the results of qPCR analysis of pro apoptosis and anti-apoptosis markers in Panc-1 cells. BAX is significantly elevated, and BCl-2 is decreased 1 hour after exposure to BCN057 *p<0.05. The expression profile did not affect Akt-1 or Caspase-3, consistent with caspase-independent cell death.

### Example 2

### BCN057 Inhibits GSK3β Signaling to Restore Apoptosis in PANC1 Cells

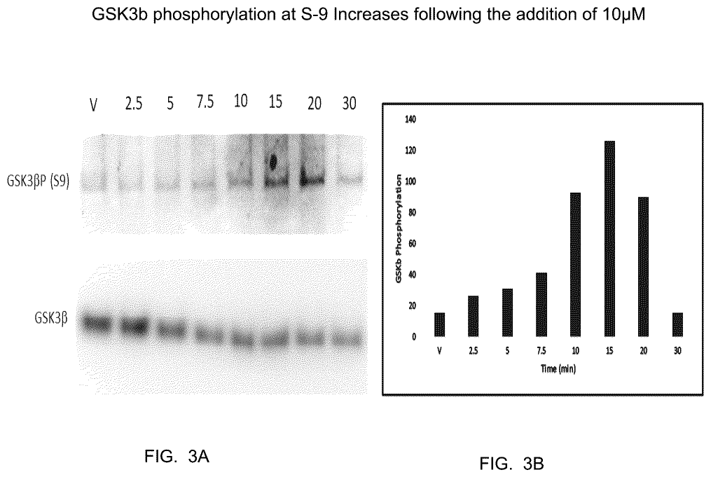

The Panc1 cell line possesses a KRASG12D mutation and has lost the capacity for apoptosis, which is a hallmark of oncogenic RAS mutations. To investigate the role BCN057 may have in the restoration of apoptosis in this cell line, the GSK3b-c-Jun relationship was assessed as it has been directly linked to apoptosis. Others have reported a link with GSK3b and c-Jun and apoptosis along with the fact that X is over expressed in the oncogenic KRAS phenotype. Analysis of Panc1 cells for GSK3b inhibition over time after exposure to 10 μM BCN057 showed rapid induction of phosphorylation at S-9 (FIG. 3A and FIG. 3B). This inhibition was noted among other non-transformed cell types such as HEK298.

Phosphorylation at T 91/93 in c-Jun was measured as a function of total c-Jun. Significant phosphorylation at T 91/93 was observed which has been reported to be associated with the activation of c-Jun's pro-apoptotic activity. This activity was not found to be induced in non-transformed cells when treated with BCN057 or even non-transformed HEK298 epithelial cells (data not shown).

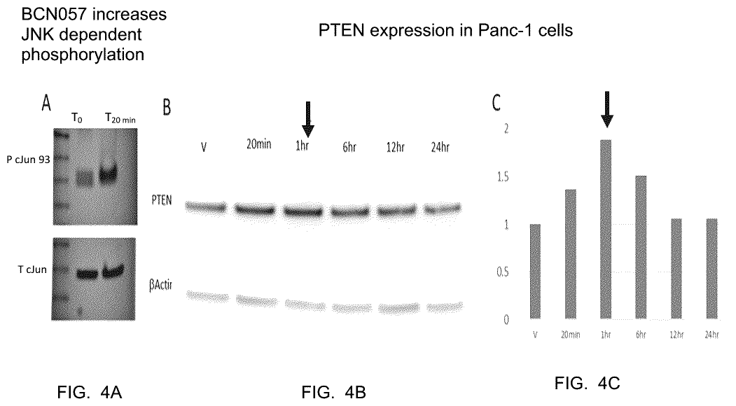

In KRAS dependent tumors, c-Jun acts to suppress Phosphatase and tensin homolog (PTEN) expression at the promoter level and that suppression of PTEN expression is essential for anti-apoptosis and cellular transformation by oncogenic RAS. Thus, we investigated PTEN expression levels in response to BCN057. Consistent with the finding of c-Jun pro-apoptotic activation, under BCN057 treatment of the cells, we found an increased expression up to 1 hr of PTEN protein (FIG. 4B and FIG. 4C) and trailing decrease thereafter, concurrent with our findings of cellular apoptosis (FIG. 2).

FIG. 4A is a western blot of Phospho-c-Jun (T-91/93). BCN057 significantly increases JNK dependent phosphorylation in KRAS mutant Panc-1 cancer cells within 20 minutes after drug treatment. Phosphorylation of c-Jun at T-91/93 is known to induce apoptosis. Total c-Jun protein level was unchanged. FIG. 4B shows PTEN expression in Panc-1 cells. Specifically, a western blot of PTEN demonstrates protein expression over 24 hours with bActin stain from the same sample with vehicle or BCN057 treatment (10 μM) at the indicated time points. FIG. 4C is a histogram of the ratio of PTEN to bActin densitometry showing the fold increase in PTEN level in B. Arrows indicate the onset of apoptosis.

### Example 3

### BCN057 Augments Anti-Neoplastic Effect of Chemotherapeutic Agents

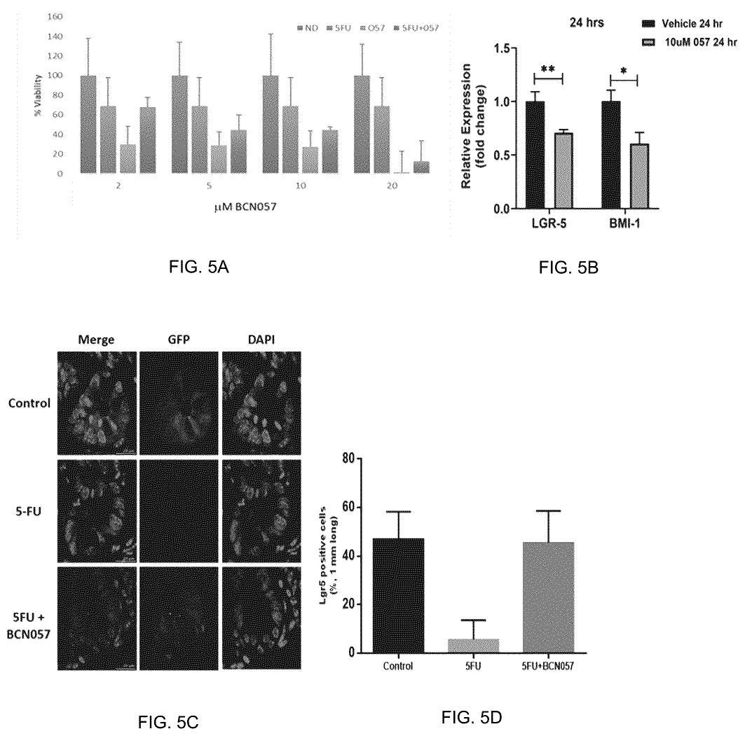

The anti-apoptotic phenotype of KRAS is associated with significant resistance to chemotherapy. Thus, to examine whether BCN057 enhances cytotoxic chemotherapeutic agents such as 5-Fluorouracil, also known as 5FU, Panc1 cells were pre-treated with 5FU as reported and incubated with BCN057. A combination of 5FU and BCN057 at equal concentrations and conditions showed greater cytotoxicity than 5FU alone (FIG. 5A). The effect followed a dose dependent response for BCN057 and was consistent over 24, 48 and 72 hrs after BN057 treatment (24 and 48 hrs not shown).

FIG. 5A shows the effects of BCN057 and 5-FU on the survival of pancreatic cancer cell lines as assessed by ATP Lite™ assay (Perkin Elmer) with incubation of all drugs at 72 hrs. All data points are n=5. Control (ND) received no drugs. Study groups include 5FU (50 μM), 057 (BCN057 at 2, 5, 10, and 20 μM or 5FU+BCN057 (BCN057 at 2, 5, 10, and 20 μM in combination with 5FU at 50 μM). The Percent viability was calculated by dividing the average value for each condition by the average value of the control cells (V) with standard deviation at each time point

LGR5 and BM/1 in pancreatic cancer: LGR5 has showed wide-spread expression in all cells of pancreatic tumors. Others have implemented BMI1 as a key contributor to chemoresistance and invasiveness which is measurable in Panc-1. We tested if BCN057 caused an increase in these cell populations in Panc-1 cells and found no significant increase in the abundance (FIG. 5B) upon dosing with 10 μm.

Epithelial LGR5 Stem cells: Previous reports from our group as well as others demonstrated that Lgr5+ve crypt base columnar cells are sensitive to chemoradiation therapy. We have also reported that protection of Lgr5+ve ISCs from treatment induced toxicity is crucial to minimize intestinal epithelial damage. 5FU is a component of FOLFIRINOX and the active agent metabolized from Capecitabine (Xeloda®). To, examine the effect of BCN057 against chemo induced toxicity in Lgr5+ ISCs, Lgr5-Cre-ERT-GFP mice were pre-treated with 5FU as reported and then subjected to BCN057 treatment. Confocal microscopic imaging of jejunal section clearly showed significant loss of Lgr5+ ISCs in 5FU treated mice. However, 5FU treated mice receiving BCN057 demonstrated significant presence of Lgr5+ve ISCs (FIG. 5C and FIG. 5D) suggesting BCN057 protects ISCs from chemotoxicity similar to radiation.

FIG. 5C demonstrates that BCN057 protects GI epithelium and the LGR5 stem cells (green) from 5FU cytotoxicity. Stains of GI crypts with green=LGR5+ stem cells. Mice were treated with 5FU and 5FU+BCN057 for 5 days. BCN057 was administered for two extra days after 5FU treatment. Small intestine samples were harvested and stained for LGR5 at 3 days after the last 5FU injection. FIG. 5D is a histogram of the quantitation by % of LGR5+ cells remaining in 1 mM sections at the crypt base showing prevention of 5-FU induced apoptosis in LGR5+ stem cells in GI.

Combined chemotherapy and cytotoxicity: Other cytotoxic drugs commonly used in the treatment of pancreatic or colorectal cancer were then examined in similar fashion such as those within FOLFIRINOX such as 5FU, Irinotecan and Oxaliplatin in combination equivalent doses of BCN057 over 24, 48 and 72 hours. In each case, the restoration of apoptosis induced statistically significant sensitivity to the cytotoxic effect of the drugs or combinations with BCN057 in all time point except one (Irinotecan (IRI) and oxaliplatin (Ox) at 72 hours) post treatment.

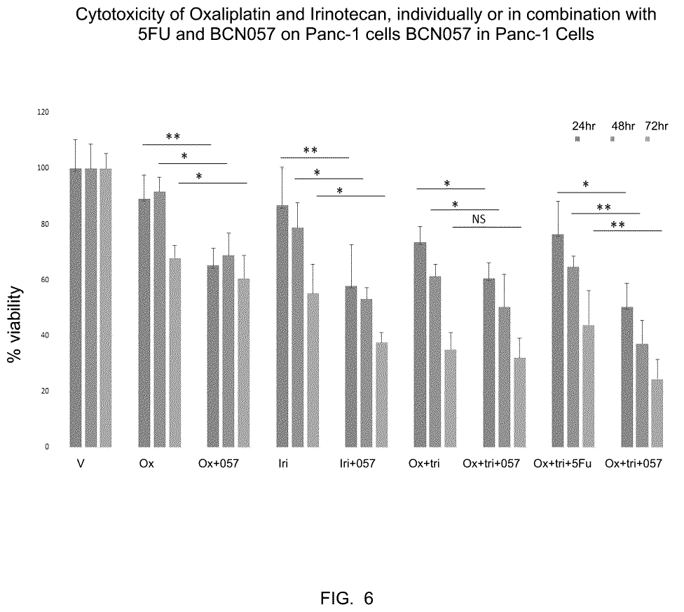

FIG. 6 is a histogram showing the cytotoxicity of Oxaliplatin and Irinotecan, individually or in combination with 5FU and BCN057 on Panc-1 cells. Cells were incubated with Oxaliplatin, Irinotecan and 5FU with concentrations at 10 μM each and BCN057 (057) also at 10 μM with n=4 for all assays. In all cases, the addition of BCN057 resulted in a general trend of decreasing tumor viability over 72 hrs. *p<0.05 **p<0.005.

This trend remained the same with a separate study using much higher doses of Oxaliplatin (75 μM) Irinotecan and 5FU concentrations at 50 μM each and BCN057 (057) remaining at 10 μM with n=6 (data not shown) and is consistent with the observation of others.

BCN057 and Immune Checkpoint blockade: An emerging method of treatment for many forms of cancer, including PDAC, is immune checkpoint inhibitors. Because GSK3b is a key regulator of immune checkpoints and in particular the PD-1/PDL-1 axis, we sought to examine the effect of BCN057 on PD-1/PDL1 expression. A co-culture system consisting of Panc1 cells and human T cells separated by a membrane was used as previously described (Neubert, N. J. et al., Frontiers in Immunology 7, 2016). Panc-1 cells were co cultured with T-Cells for one day and subsequently treated with BCN057 at 10 μM for up to 12 hours.

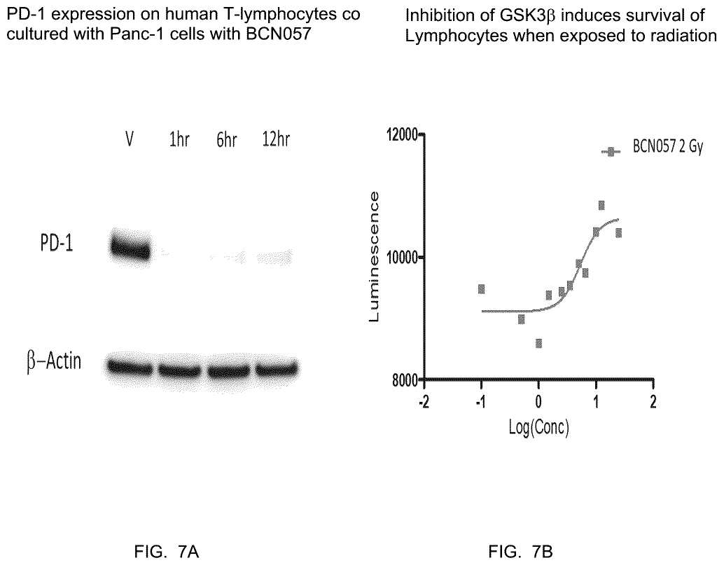

FIG. 7A is a western blot showing PD-1 expression on human T-lymphocytes co-cultured with Panc-1 cells with no drug (vehicle) or 1, 6 and 12 hours with BCN057. FIG. 7B is a graph showing that the inhibition of GSK3b induces survival of Lymphocytes when exposed to radiation. Invitro viability assay using increasing concentrations of BCN057 on irradiated mouse T-lymphocytes EC₅₀=5.34 mM which is within the plasma concentrations observed for the drug.

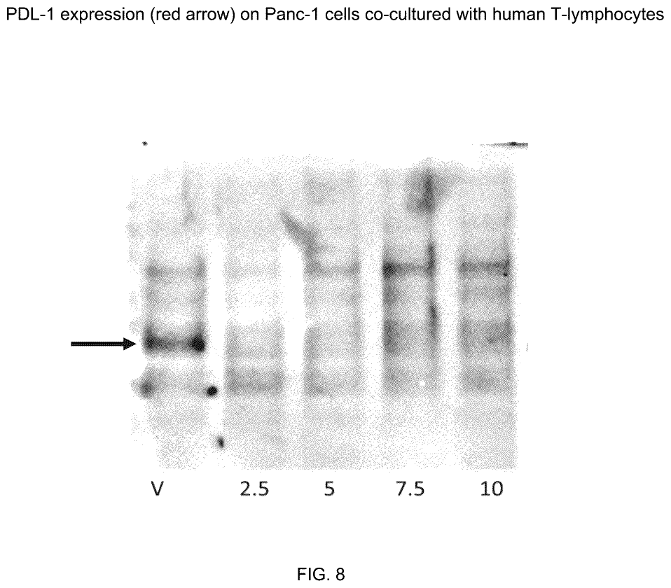

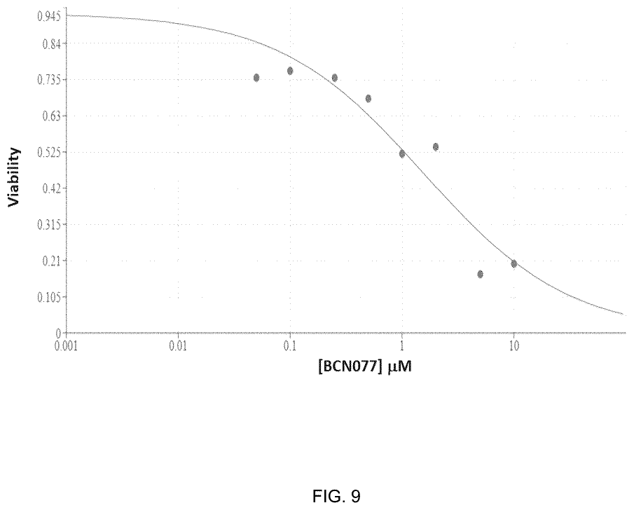

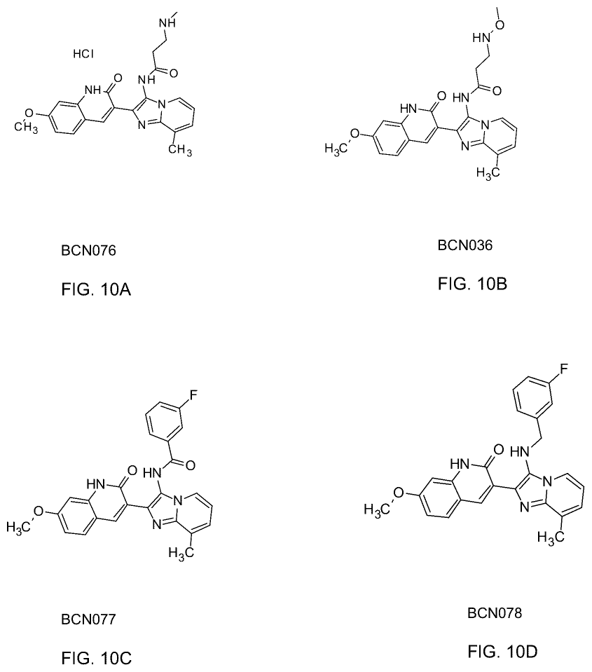

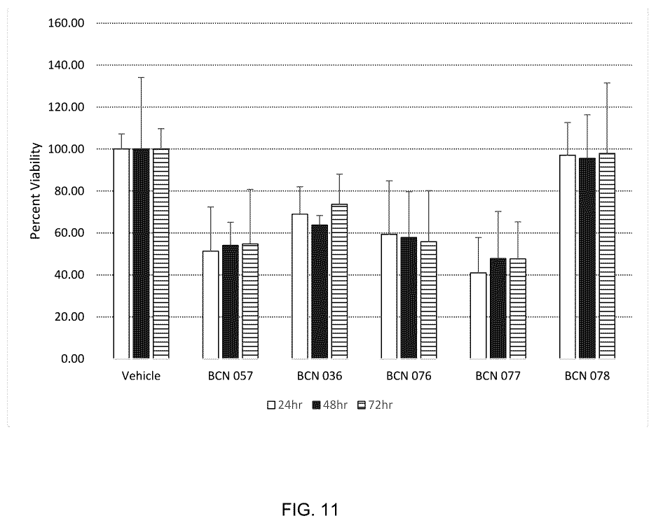

FIG. 8 is western blot showing PDL-1 expression (red arrow) on Panc-1 cells co cultured with human T-lymphocytes with no drug (vehicle) or 2.5, 5, 7.5 and 10 hrs. with BCN057. PDL-1 expression on Panc-1 cells was not appreciably changed, however, the cells were undergoing rapid apoptosis as described. However, PD-1 expression on human T-lymphocytes were decreased significantly within the first hour and sustained over the 12 hours post treatment as shown in FIG. 7A. In a separate study using mouse T-lymphocytes, 2Gy radiation was used to induce apoptosis with increasing amounts of BCN057. The results demonstrated the preservation of the T-lymphocytes by BCN057 in the presence of radiation (FIG. 7B) with an EC₅₀=5.34 μM, thus establishing the difference in biological response between the tumor and immune cells.

In summary the results demonstrate that BCN057 can selectively enhance cytotoxic effect of chemotherapeutic agents in oncogenic KRAS pancreatic cancer cells. Moreover, it mitigates chemo-toxic effect in normal intestinal epithelium suggesting that BCN057 can be considered as an adjuvant therapy to improve therapeutic index. As an agent that can restore apoptosis in the oncogenic RAS phenotype, this aligns well with a use case for inducing greater tumor sensitivity to chemotherapeutic agents. In a previous publication (Bhanja, P., et. al., Stem Cell Research & Therapy 9, 2018) we demonstrated a model in mice using the syngeneic KRASG13R mutant MC38 tumor cell line where mice receiving BCN057 had significantly lower tumor burden than control, however, mice with receiving BCN057 with radiation treatment had lower tumor burden and yet were able to tolerate much higher radiation doses than those receiving radiation alone. This highlights the value of targeting pathways which are wired very differently in the transformed vs. non-transformed phenotype where this dual activity in tumoricidal as well as normal tissue sparing activity may lead to better overall and progression free survival.

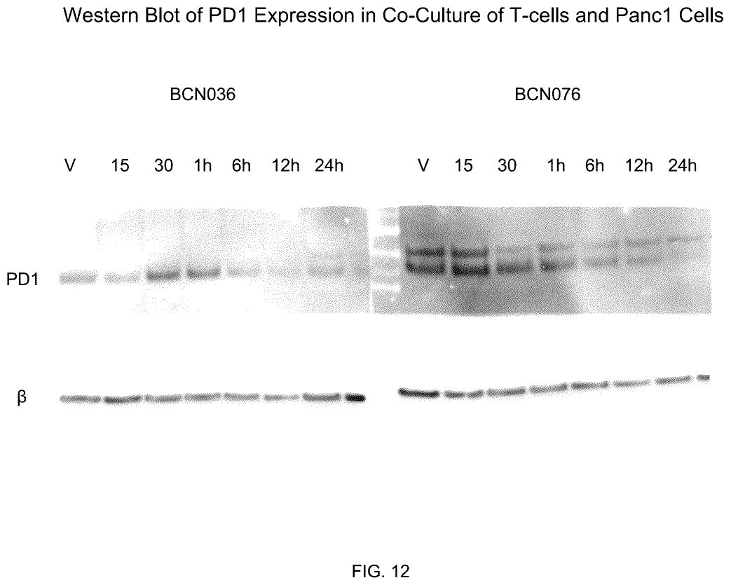

FIG. 12 is a western blot of PD1 Expression in Co-Culture of T-cells and Panc1 cells. Samples were tested over time (i.e., at 15 minutes, 30 minutes. 1 hour, 6 hours, 12 hours and 24 hours). The results demonstrate that both BCN036 and BCN076 inhibit PD1 expression over time. Beta-actin (β) was used as a control.

### Pharmaceutical Carriers and Administration

The present methods may prevent a disease or condition or one or more symptoms of a disease or condition. As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The compositions and methods of the present disclosure may be utilized to treat an individual in need thereof. In certain embodiments, the individual is a mammal such as a human, or a non-human mammal. When administered to an animal, such as a human, the composition or the compound is preferably administered or used as a pharmaceutical composition comprising, for example, a compound of the disclosure and a pharmaceutically acceptable carrier.

Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophilized for reconstitution, powder, solution, syrup, suppository, injection or the like. The composition can also be present in a transdermal delivery system, e.g., a skin patch.

A pharmaceutical composition disclosed herein may comprise a therapeutic compound in an amount sufficient to allow customary administration to an individual. In certain embodiments, a pharmaceutical composition disclosed herein may comprise, e.g., at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of a therapeutic compound. In certain embodiments, a pharmaceutical composition disclosed herein may comprise, e.g., at least 5 mg, at least 10 mg, at least 20 mg, at least 25 mg, at least 50 mg, at least 75 mg, at least 100 mg, at least 200 mg, at least 300 mg, at least 400 mg, at least 500 mg, at least 600 mg, at least 700 mg, at least 800 mg, at least 900 mg, at least 1,000 mg, at least 1,100 mg, at least 1,200 mg, at least 1,300 mg, at least 1,400 mg, or at least 1,500 mg of a therapeutic compound. In yet other aspects of this embodiment, a pharmaceutical composition disclosed herein may comprise in the range of, e.g., about 5 mg to about 100 mg, about 10 mg to about 100 mg, about 50 mg to about 150 mg, about 100 mg to about 250 mg, about 150 mg to about 350 mg, about 250 mg to about 500 mg, about 350 mg to about 600 mg, about 500 mg to about 750 mg, about 600 mg to about 900 mg, about 750 mg to about 1,000 mg, about 850 mg to about 1,200 mg, or about 1,000 mg to about 1,500 mg. In still certain embodiments, a pharmaceutical composition disclosed herein may comprise in the range of, e.g., about 10 mg to about 250 mg, about 10 mg to about 500 mg, about 10 mg to about 750 mg, about 10 mg to about 1,000 mg, about 10 mg to about 1,500 mg, about 50 mg to about 250 mg, about 50 mg to about 500 mg, about 50 mg to about 750 mg, about 50 mg to about 1,000 mg, about 50 mg to about 1,500 mg, about 100 mg to about 250 mg, about 100 mg to about 500 mg, about 100 mg to about 750 mg, about 100 mg to about 1,000 mg, about 100 mg to about 1,500 mg, about 200 mg to about 500 mg, about 200 mg to about 750 mg, about 200 mg to about 1,000 mg, about 200 mg to about 1,500 mg, about 5 mg to about 1,500 mg, about 5 mg to about 1,000 mg, or about 5 mg to about 250 mg.

A pharmaceutical composition disclosed herein may comprise a solvent, emulsion or other diluent in an amount sufficient to dissolve a therapeutic compound disclosed herein. In certain embodiments, a pharmaceutical composition disclosed herein may comprise a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In certain embodiments, a pharmaceutical composition disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

The final concentration of a therapeutic compound disclosed herein in a pharmaceutical composition disclosed herein may be of any suitable concentration. In certain embodiments, the final concentration of a therapeutic compound in a pharmaceutical composition may be a therapeutically effective amount. In certain embodiments, the final concentration of a therapeutic compound in a pharmaceutical composition may be, e.g., at least 0.00001 mg/mL, at least 0.0001 mg/mL, at least 0.001 mg/mL, at least 0.01 mg/mL, at least 0.1 mg/mL, at least 1 mg/mL, at least 10 mg/mL, at least 25 mg/mL, at least 50 mg/mL, at least 100 mg/mL, at least 200 mg/mL, at least 500 mg/mL, at least 700 mg/mL, at least 1,000 mg/mL, or at least 1,200 mg/mL. In certain embodiments, the concentration of a therapeutic compound disclosed herein in the solution may be, e.g., at most 1,000 mg/mL, at most 1,100 mg/mL, at most 1,200 mg/mL, at most 1,300 mg/mL, at most 1,400 mg/mL, at most 1,500 mg/mL, at most 2,000 mg/mL, at most 2,000 mg/mL, or at most 3,000 mg/mL. In certain embodiments, the final concentration of a therapeutic compound in a pharmaceutical composition may be in a range of, e.g., about 0.00001 mg/mL to about 3,000 mg/mL, about 0.0001 mg/mL to about 3,000 mg/mL, about 0.01 mg/mL to about 3,000 mg/mL, about 0.1 mg/mL to about 3,000 mg/mL, about 1 mg/mL to about 3,000 mg/mL, about 250 mg/mL to about 3,000 mg/mL, about 500 mg/mL to about 3,000 mg/mL, about 750 mg/mL to about 3,000 mg/mL, about 1,000 mg/mL to about 3,000 mg/mL, about 100 mg/mL to about 2,000 mg/mL, about 250 mg/mL to about 2,000 mg/mL, about 500 mg/mL to about 2,000 mg/mL, about 750 mg/mL to about 2,000 mg/mL, about 1,000 mg/mL to about 2,000 mg/mL, about 100 mg/mL to about 1,500 mg/mL, about 250 mg/mL to about 1,500 mg/mL, about 500 mg/mL to about 1,500 mg/mL, about 750 mg/mL to about 1,500 mg/mL, about 1,000 mg/mL to about 1,500 mg/mL, about 100 mg/mL to about 1,200 mg/mL, about 250 mg/mL to about 1,200 mg/mL, about 500 mg/mL to about 1,200 mg/mL, about 750 mg/mL to about 1,200 mg/mL, about 1,000 mg/mL to about 1,200 mg/mL, about 100 mg/mL to about 1,000 mg/mL, about 250 mg/mL to about 1,000 mg/mL, about 500 mg/mL to about 1,000 mg/mL, about 750 mg/mL to about 1,000 mg/mL, about 100 mg/mL to about 750 mg/mL, about 250 mg/mL to about 750 mg/mL, about 500 mg/mL to about 750 mg/mL, about 100 mg/mL to about 500 mg/mL, about 250 mg/mL to about 500 mg/mL, about 0.00001 mg/mL to about 0.0001 mg/mL, about 0.00001 mg/mL to about 0.001 mg/mL, about 0.00001 mg/mL to about 0.01 mg/mL, about 0.00001 mg/mL to about 0.1 mg/mL, about 0.00001 mg/mL to about 1 mg/mL, about 0.001 mg/mL to about 0.01 mg/mL, about 0.001 mg/mL to about 0.1 mg/mL, about 0.001 mg/mL to about 1 mg/mL, about 0.001 mg/mL to about 10 mg/mL, or about 0.001 mg/mL to about 100 mg/mL.

In certain embodiments, a therapeutically effective amount of a therapeutic compound disclosed herein generally is in the range of about 0.001 mg/kg/day to about 100 mg/kg/day. In certain embodiments, an effective amount of a therapeutic compound disclosed herein may be, e.g., at least 0.001 mg/kg/day, at least 0.01 mg/kg/day, at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, or at least 50 mg/kg/day. In certain embodiments, an effective amount of a therapeutic compound disclosed herein may be in the range of, e.g., about 0.001 mg/kg/day to about 10 mg/kg/day, about 0.001 mg/kg/day to about 15 mg/kg/day, about 0.001 mg/kg/day to about 20 mg/kg/day, about 0.001 mg/kg/day to about 25 mg/kg/day, about 0.001 mg/kg/day to about 30 mg/kg/day, about 0.001 mg/kg/day to about 35 mg/kg/day, about 0.001 mg/kg/day to about 40 mg/kg/day, about 0.001 mg/kg/day to about 45 mg/kg/day, about 0.001 mg/kg/day to about 50 mg/kg/day, about 0.001 mg/kg/day to about 75 mg/kg/day, about 0.001 mg/kg/day to about 100 mg/kg/day, about 0.001 mg/kg/day to about 150 mg/kg/day, about 0.001 mg/kg/day to about 200 mg/kg/day, about 0.001 mg/kg/day to about 250 mg/kg/day, about 0.001 mg/kg/day to about 300 mg/kg/day, about 0.001 mg/kg/day to about 350 mg/kg/day, about 0.001 mg/kg/day to about 400 mg/kg/day, about 0.001 mg/kg/day to about 450 mg/kg/day, about 0.001 mg/kg/day to about 500 mg/kg/day, about 0.001 mg/kg/day to about 550 mg/kg/day, about 0.001 mg/kg/day to about 600 mg/kg/day, about 0.001 mg/kg/day to about 650 mg/kg/day, about 0.001 mg/kg/day to about 700 mg/kg/day, about 0.001 mg/kg/day to about 750 mg/kg/day, or about 0.001 mg/kg/day to about 800 mg/kg/day. In yet other aspects of this embodiment, an effective amount of a therapeutic compound disclosed herein may be in the range of, e.g., about 0.01 mg/kg/day to about 10 mg/kg/day, about 0.01 mg/kg/day to about 15 mg/kg/day, about 0.01 mg/kg/day to about 20 mg/kg/day, about 0.01 mg/kg/day to about 25 mg/kg/day, about 0.01 mg/kg/day to about 30 mg/kg/day, about 0.01 mg/kg/day to about 35 mg/kg/day, about 0.01 mg/kg/day to about 40 mg/kg/day, about 0.01 mg/kg/day to about 45 mg/kg/day, about 0.01 mg/kg/day to about 50 mg/kg/day, about 0.01 mg/kg/day to about 75 mg/kg/day, about 0.01 mg/kg/day to about 100 mg/kg/day, about 0.01 mg/kg/day to about 150 mg/kg/day, about 0.01 mg/kg/day to about 200 mg/kg/day, about 0.01 mg/kg/day to about 250 mg/kg/day, about 0.01 mg/kg/day to about 300 mg/kg/day, about 0.01 mg/kg/day to about 350 mg/kg/day, about 0.01 mg/kg/day to about 400 mg/kg/day, about 0.01 mg/kg/day to about 450 mg/kg/day, about 0.01 mg/kg/day to about 500 mg/kg/day, about 0.01 mg/kg/day to about 550 mg/kg/day, about 0.01 mg/kg/day to about 600 mg/kg/day, about 0.01 mg/kg/day to about 650 mg/kg/day, about 0.01 mg/kg/day to about 700 mg/kg/day, about 0.01 mg/kg/day to about 750 mg/kg/day, or about 0.01 mg/kg/day to about 800 mg/kg/day. In certain embodiments, an effective amount of a therapeutic compound disclosed herein may be in the range of, e.g., about 0.1 mg/kg/day to about 10 mg/kg/day, about 0.1 mg/kg/day to about 15 mg/kg/day, about 0.1 mg/kg/day to about 20 mg/kg/day, about 0.1 mg/kg/day to about 25 mg/kg/day, about 0.1 mg/kg/day to about 30 mg/kg/day, about 0.1 mg/kg/day to about 35 mg/kg/day, about 0.1 mg/kg/day to about 40 mg/kg/day, about 0.1 mg/kg/day to about 45 mg/kg/day, about 0.1 mg/kg/day to about 50 mg/kg/day, about 0.1 mg/kg/day to about 75 mg/kg/day, about 0.1 mg/kg/day to about 100 mg/kg/day, about 0.1 mg/kg/day to about 150 mg/kg/day, about 0.1 mg/kg/day to about 200 mg/kg/day, about 0.1 mg/kg/day to about 250 mg/kg/day, about 0.1 mg/kg/day to about 300 mg/kg/day, about 0.1 mg/kg/day to about 350 mg/kg/day, about 0.1 mg/kg/day to about 400 mg/kg/day, about 0.1 mg/kg/day to about 450 mg/kg/day, about 0.1 mg/kg/day to about 500 mg/kg/day, about 0.1 mg/kg/day to about 550 mg/kg/day, about 0.1 mg/kg/day to about 600 mg/kg/day, about 0.1 mg/kg/day to about 650 mg/kg/day, about 0.1 mg/kg/day to about 700 mg/kg/day, about 0.1 mg/kg/day to about 750 mg/kg/day, or about 0.1 mg/kg/day to about 800 mg/kg/day. In certain embodiments, an effective amount of a therapeutic compound disclosed herein may be in the range of, e.g., about 10 mg/kg/day to about 15 mg/kg/day, about 10 mg/kg/day to about 20 mg/kg/day, about 10 mg/kg/day to about 25 mg/kg/day, about 10 mg/kg/day to about 30 mg/kg/day, about 10 mg/kg/day to about 35 mg/kg/day, about 10 mg/kg/day to about 40 mg/kg/day, about 10 mg/kg/day to about 45 mg/kg/day, about 10 mg/kg/day to about 50 mg/kg/day, about 10 mg/kg/day to about 75 mg/kg/day, about 10 mg/kg/day to about 100 mg/kg/day, about 10 mg/kg/day to about 150 mg/kg/day, about 10 mg/kg/day to about 200 mg/kg/day, about 10 mg/kg/day to about 250 mg/kg/day, about 10 mg/kg/day to about 300 mg/kg/day, about 10 mg/kg/day to about 350 mg/kg/day, about 10 mg/kg/day to about 400 mg/kg/day, about 10 mg/kg/day to about 450 mg/kg/day, about 10 mg/kg/day to about 500 mg/kg/day, about 10 mg/kg/day to about 550 mg/kg/day, about 10 mg/kg/day to about 600 mg/kg/day, about 10 mg/kg/day to about 650 mg/kg/day, about 10 mg/kg/day to about 700 mg/kg/day, about 10 mg/kg/day to about 750 mg/kg/day, or about 10 mg/kg/day to about 800 mg/kg/day.

In other aspects of this embodiment, an effective amount of a therapeutic compound disclosed herein may be in the range of, e.g., about 1 mg/kg/day to about 10 mg/kg/day, about 1 mg/kg/day to about 15 mg/kg/day, about 1 mg/kg/day to about 20 mg/kg/day, about 1 mg/kg/day to about 25 mg/kg/day, about 1 mg/kg/day to about 30 mg/kg/day, about 1 mg/kg/day to about 35 mg/kg/day, about 1 mg/kg/day to about 40 mg/kg/day, about 1 mg/kg/day to about 45 mg/kg/day, about 1 mg/kg/day to about 50 mg/kg/day, about 1 mg/kg/day to about 75 mg/kg/day, or about 1 mg/kg/day to about 100 mg/kg/day. In certain embodiments, an effective amount of a therapeutic compound disclosed herein may be in the range of, e.g., about 5 mg/kg/day to about 10 mg/kg/day, about 5 mg/kg/day to about 15 mg/kg/day, about 5 mg/kg/day to about 20 mg/kg/day, about 5 mg/kg/day to about 25 mg/kg/day, about 5 mg/kg/day to about 30 mg/kg/day, about 5 mg/kg/day to about 35 mg/kg/day, about 5 mg/kg/day to about 40 mg/kg/day, about 5 mg/kg/day to about 45 mg/kg/day, about 5 mg/kg/day to about 50 mg/kg/day, about 5 mg/kg/day to about 75 mg/kg/day, or about 5 mg/kg/day to about 100 mg/kg/day.

In liquid and semi-solid formulations, a concentration of a therapeutic compound disclosed herein typically may be between about 50 mg/mL to about 1,000 mg/mL. In certain embodiments, a therapeutically effective amount of a therapeutic disclosed herein may be from, e.g., about 50 mg/mL to about 100 mg/mL, about 50 mg/mL to about 200 mg/mL, about 50 mg/mL to about 300 mg/mL, about 50 mg/mL to about 400 mg/mL, about 50 mg/mL to about 500 mg/mL, about 50 mg/mL to about 600 mg/mL, about 50 mg/mL to about 700 mg/mL, about 50 mg/mL to about 800 mg/mL, about 50 mg/mL to about 900 mg/mL, about 50 mg/mL to about 1,000 mg/mL, about 100 mg/mL to about 200 mg/mL, about 100 mg/mL to about 300 mg/mL, about 100 mg/mL to about 400 mg/mL, about 100 mg/mL to about 500 mg/mL, about 100 mg/mL to about 600 mg/mL, about 100 mg/mL to about 700 mg/mL, about 100 mg/mL to about 800 mg/mL, about 100 mg/mL to about 900 mg/mL, about 100 mg/mL to about 1,000 mg/mL, about 200 mg/mL to about 300 mg/mL, about 200 mg/mL to about 400 mg/mL, about 200 mg/mL to about 500 mg/mL, about 200 mg/mL to about 600 mg/mL, about 200 mg/mL to about 700 mg/mL, about 200 mg/mL to about 800 mg/mL, about 200 mg/mL to about 900 mg/mL, about 200 mg/mL to about 1,000 mg/mL, about 300 mg/mL to about 400 mg/mL, about 300 mg/mL to about 500 mg/mL, about 300 mg/mL to about 600 mg/mL, about 300 mg/mL to about 700 mg/mL, about 300 mg/mL to about 800 mg/mL, about 300 mg/mL to about 900 mg/mL, about 300 mg/mL to about 1,000 mg/mL, about 400 mg/mL to about 500 mg/mL, about 400 mg/mL to about 600 mg/mL, about 400 mg/mL to about 700 mg/mL, about 400 mg/mL to about 800 mg/mL, about 400 mg/mL to about 900 mg/mL, about 400 mg/mL to about 1,000 mg/mL, about 500 mg/mL to about 600 mg/mL, about 500 mg/mL to about 700 mg/mL, about 500 mg/mL to about 800 mg/mL, about 500 mg/mL to about 900 mg/mL, about 500 mg/mL to about 1,000 mg/mL, about 600 mg/mL to about 700 mg/mL, about 600 mg/mL to about 800 mg/mL, about 600 mg/mL to about 900 mg/mL, or about 600 mg/mL to about 1,000 mg/mL.

The subject may be a human, rat, mouse, cat, dog, horse, sheep, cow, monkey, avian, or amphibian. In another embodiment, the cell is in vivo or in vitro. Typical subjects to which compounds of the disclosure may be administered will be mammals, particularly primates, especially humans. For veterinary applications, a wide variety of subjects will be suitable, e. g. livestock such as cattle, sheep, goats, cows, swine and the like; poultry such as chickens, ducks, geese, turkeys, and the like; and domesticated animals particularly pets such as dogs and cats. For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids and cell samples of the above subjects will be suitable for use such as mammalian, particularly primate such as human, blood, urine or tissue samples, or blood urine or tissue samples of the animals mentioned for veterinary applications.

When administering to an organism, the compound may be administered by any suitable means. In some embodiments, the compounds or formulations are administered orally. In some embodiments, the compounds or formulations are administered by injection, e.g., subcutaneous, parenteral, or intravenous, injections.

In some embodiments the compound may be administered in combination with other potential mitigators. In a particular embodiment, the composition may be administered with growth factors, NSAIDs, chemotherapeutics, anti-inflammatories, antibiotics, Metformin (Glucophage, Glumetza, others), Sulfonylureas, Meglitinides, Thiazolidinediones, DPP-4 inhibitors, GLP-1 receptor agonists, SGLT2 inhibitors, and/or Insulin therapy, for the treatment of the above conditions. In one aspect, the growth factor can be G-CSF (aka filgrastim, NEUPOGEN®) or erythropoietin (aka EPOGEN®).

In other embodiments, the compositions may comprise an effective amount of a modulator and/or other pharmaceutically active agent in a physiologically-acceptable carrier. The carrier may take a wide variety of forms depending on the form of preparation desired for a particular route of administration. Suitable carriers and their formulation are described, for example, in Remington's Pharmaceutical Sciences by E. W. Martin. In some embodiments, the compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) or oral administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

In some embodiments, the compositions may be in a form suitable for administration by sterile injection. In one example, to prepare such a composition, the compositions(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). For parenteral formulations, the carrier will usually comprise sterile water, though other ingredients, for example, ingredients that aid solubility or for preservation, may be included. Injectable solutions may also be prepared in which case appropriate stabilizing agents may be employed. In one embodiment, the formulation includes at least one or more of methanesulfonic acid, povidone, benzyl alcohol, n-Methyl pyrrolidone, ethaonol, Poloxamer 188, lactic acid, Captisol (SBE-beta-CD), or Vitamin E, such as TPGS (d-alpha tocopheryl polyethylene glycol 1000 succinate).

Formulations suitable for parenteral administration usually comprise a sterile aqueous preparation of the compound, which may be isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery or localized delivery. Administration may for example be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

In some embodiments, the compositions may be in a form suitable for oral administration. In compositions in oral dosage form, any of the usual pharmaceutical media may be employed. Thus, for liquid oral preparations, such as, for example, suspensions, elixirs and solutions, suitable carriers and additives include water, glycols, oils, alcohols, flavoring agents, preservatives, coloring agents and the like. For solid oral preparations such as, for example, powders, capsules and tablets, suitable carriers and additives include starches, sugars, diluents, granulating agents, lubricants, binders, disintegrating agents and the like. If desired, tablets may be sugar coated or enteric coated by standard techniques.

Compositions suitable for oral administration may be presented as discrete units such as capsules, cachets, tablets, or lozenges, each containing a predetermined amount of the active ingredient as a powder or granules. Optionally, a suspension in an aqueous liquor or a non-aqueous liquid may be employed, such as a syrup, an elixir, an emulsion, or a draught. Formulations for oral use include tablets containing active ingredient(s) in a mixture with pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

A syrup can be made by adding the compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

In some embodiments, the composition can be in a form of nasal or other mucosal spray formulations (e.g. inhalable forms). These formulations can include purified aqueous solutions of the active compounds with preservative agents and isotonic agents. Such formulations can be adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

In some embodiments, the composition may be in a form suitable for rectal administration. These formulations may be presented as a suppository with a suitable carrier such as cocoa butter, hydrogenated fats, or hydrogenated fatty carboxylic acids.

In some embodiments, the composition may be in a form suitable for transdermal administration. These formulations may be prepared, for example, by incorporating the active compound in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

In addition to the aforementioned ingredients, compositions of the disclosure may further include one or more accessory ingredient(s) selected from encapsulants, diluents, buffers, flavoring agents, binders, disintegrants, surface active agents, thickeners, lubricants, preservatives (including antioxidants), and the like.

In some embodiments, compositions may be formulated for immediate release, sustained release, delayed-onset release or any other release profile known to one skilled in the art. In some embodiments, the pharmaceutical composition may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that target the site of a pathology. For some applications, controlled release formulations obviate the need for frequent dosing to sustain activity at a medically advantageous level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound is formulated with appropriate excipients into a pharmaceutical composition that, upon administration, releases the compound in a controlled manner. Examples include single or multiple unit tablet or capsule compositions, oil solutions, suspensions, emulsions, microcapsules, microspheres, molecular complexes, nanoparticles, patches, and liposomes.

In some embodiments, the composition may comprise a “vectorized” form, such as by encapsulation of the compound in a liposome or other encapsulate medium, or by fixation of the compound, e.g., by covalent bonding, chelation, or associative coordination, on a suitable biomolecule, such as those selected from proteins, lipoproteins, glycoproteins, and polysaccharides.

In some embodiments, the composition can be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents. Alternatively, the compound may be incorporated in biocompatible carriers, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

In all embodiments, the compound or other active compounds may be present as pharmaceutically acceptable salts or other derivatives, such as ether derivatives, ester derivatives, acid derivatives, and aqueous solubility altering derivatives of the active compound. Derivatives include all individual enantiomers, diastereomers, racemates, and other isomers of the compounds. Derivatives also include all polymorphs and solvates, such as hydrates and those formed with organic solvents, of the compounds. Such isomers, polymorphs, and solvates may be prepared by methods known in the art, such as by regiospecific and/or enantioselective synthesis and resolution.

The ability to prepare salts depends on the acidity or basicity of the compounds. Suitable salts of the compounds include, but are not limited to, acid addition salts, such as those made with hydrochloric, hydrobromic, hydroiodic, perchloric, sulfuric, nitric, phosphoric, acetic, propionic, glycolic, lactic pyruvic, malonic, succinic, maleic, fumaric, malic, tartaric, citric, benzoic, carbonic, cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid; salts made with saccharin; alkali metal salts, such as sodium and potassium salts; alkaline earth metal salts, such as calcium and magnesium salts; and salts formed with organic or inorganic ligands, such as quaternary ammonium salts.

Additional suitable salts include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate salts of the compounds.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Unless the context clearly indicates otherwise, compositions of all embodiments can comprise various pharmaceutically acceptable salts, or other derivatives described above.

The formulation and preparation of such compositions are well known to those skilled in the art of pharmaceutical formulation. Formulations can be found in Remington: The Science and Practice of Pharmacy.

The amount of the compound employed in the present disclosure to be used varies according to the condition, the patient/subject, and the extent of the condition.

The contents of all cited references (including literature references, issued patents, published patent applications) as cited throughout this application are hereby expressly incorporated by reference. The disclosure and the manner and process of making and using it, are described in such full, clear, concise and exact terms as to enable any person skilled in the art to which it pertains, to make and use the same.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the disclosure will be that amount of the compound that is the lowest dose effective to produce a therapeutic effect. Such an effective dose will generally depend upon the factors described above.

Dosing can be single dosage or cumulative (serial dosing), and can be readily determined by one skilled in the art. For instance, treatment may comprise a one-time administration of an effective dose of a pharmaceutical composition disclosed herein. Alternatively, treatment may comprise multiple administrations of an effective dose of a pharmaceutical composition carried out over a range of time periods, such as, e.g., once daily, twice daily, thrice daily, once every few days, or once weekly. The timing of administration can vary from individual to individual, depending upon such factors as the severity of an individual's symptoms. For example, an effective dose of a pharmaceutical composition disclosed herein can be administered to an individual once daily for an indefinite period of time, or until the individual no longer requires therapy. A person of ordinary skill in the art will recognize that the condition of the individual can be monitored throughout the course of treatment and that the effective amount of a pharmaceutical composition disclosed herein that is administered can be adjusted accordingly.

If desired, the effective daily dose of the active compound may be administered as one, two, three, four, five, six or more sub-doses administered separately at appropriate intervals throughout the day, optionally, in unit dosage forms. In certain embodiments of the present disclosure, the active compound may be administered two or three times daily. In preferred embodiments, the active compound will be administered once daily.

In certain embodiments, the period of administration of a therapeutic compound is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In certain embodiments, a treatment regimen may comprise a period during which administration is stopped for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

The patient receiving this treatment is any animal in need, including primates, in particular humans, and other mammals such as equines, cattle, swine and sheep; and poultry and pets in general.

In other embodiments, the compounds described herein may be provided with the one or more additional therapeutic agents in a kit, e.g., as separate pharmaceutical formulations capable of being used together in a conjoint therapy as discussed herein, either together in a single container or in separate containers. In certain such embodiments, the kit may further include instructions for the conjoint administration of the pharmaceutical formulations, e.g., for treating or preventing any of the conditions discussed above.

Such combination products may employ compounds of this disclosure, or pharmaceutically acceptable salts thereof, within the dosage range described hereinbefore and the other pharmaceutically-active agent within its approved dosage range.

In some embodiments, the compound may be administered after the predicate event, such as after exposure to ionizing radiation, or after the initiation of exposure to radiation including accidental or therapeutic radiation. In one embodiment, the compound is administered immediately after the exposure. In another embodiment, the compound is administered within 12 hours of the exposure. In another embodiment, the compound is administered within 24 hours of the exposure. In another embodiment, the compound is administered at 24 hours after the exposure. In another embodiment, the compound is administered after 24 hours of exposure. In another embodiment, the compound is administered after 36 hours of exposure. In another embodiment, the compound is administered within 48 hours of exposure. In another embodiment, the compound is administered within 60 hours of exposure. In another embodiment, the compound is administered within 72 hours of the exposure. In another embodiment, the compound is administered within 84 hours of the exposure.

In a certain embodiment or a particular formulation Yel002/BCN057 was solubilized in aqueous solution at physiologically compatible pHs using 100 mM methanesulfonic acid (MSA)/10% povidone (PVP); 100 mM MSA/2% benzyl alcohol/2% N-methylpyrrolidone (NMP); and, 100 mM MSA/10% ethanol/1% Poloxamer 188. In a further aspect 100 mM lactic acid was added and also improved solubility for these mixtures. In yet another embodiment, a formulation comprising Yel002 and 30 wt % Captisol (SBE-beta-CD) and 100 mM MSA yielded excellent solubility at up to pH 4.1 or higher.

In another embodiment formulation for intravenous, subcutaneous and oral delivery of therapeutic levels of Yel002/BCN057 were developed comprising 30 wt % Captisol (SBE-beta-CD) and 100 mM MSA at pH 4.1 or higher (adjusted with 1.0 N NaOH).

The disclosure now being generally described, it will be more readily understood by reference to the following examples which are included merely for purposes of illustration of certain aspects and embodiments of the present disclosure and are not intended to limit the disclosure.

In other aspects of this embodiment, a molecule disclosed herein reduces the severity of a disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, a fusion protein or chimeric molecule disclosed herein reduces the severity of a disease from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the present disclosure to be practiced otherwise than specifically described herein. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

All patents, patent publications, and other publications referenced and identified in the present specification are individually and expressly incorporated herein by reference in their entirety for the purpose of describing and disclosing, for example, the compositions and methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only and is not intended to limit the scope of the present disclosure, which is defined solely by the claims. Accordingly, the present disclosure is not limited to that precisely as shown and described.

### Additional Analogs

Embodiments include a compound of Formula I:

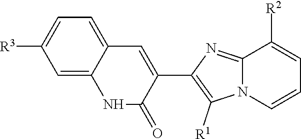

wherein R¹=an alkyl, an alkyl amine, an ether, an aryl or an aryl halide,
wherein R²=H or CH₃,
wherein R³=H, OH or OCH₃,

Embodiments include a compound of Formula I:

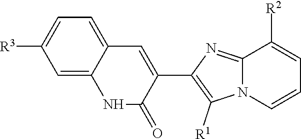

wherein R¹=

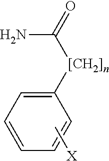

n=1, 2 or 3,
X=F, CF₃ Cl, OH or CH₂OH,
R²=H or CH₃ and R³=H, OH or OCH₃,

Embodiments include a compound of Formula I:

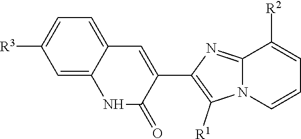

wherein R¹=

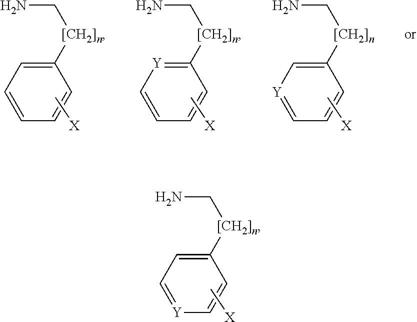

and wherein n=1, 2 or 3,
X=F, CF₃ Cl, OH or CH₂OH,
Y=O, S or N, and
R²=H or CH₃ and R³=H, OH or OCH₃.

Embodiments include a compound of Formula I:

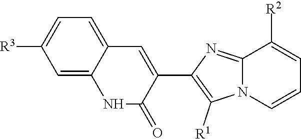

wherein R¹=

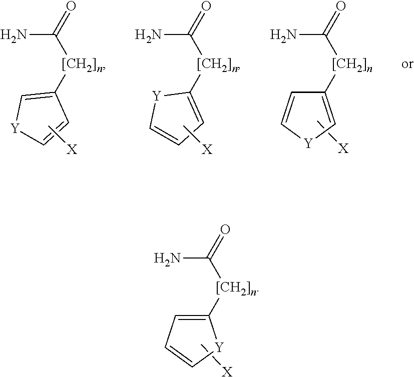

wherein n=1, 2 or 3,
X=F, CF₃ Cl, OH or CH₂OH,
Y=O, S or N,
R²=H or CH₃ and R³=H, OH or OCH₃.

Embodiments include a compound of Formula I:

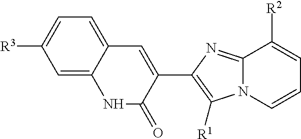

wherein R¹=

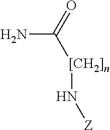

wherein n=1, 2 or 3 and
R²=H or CH₃ and R³=H, OH or OCH₃.

Embodiments include a compound of Formula I:

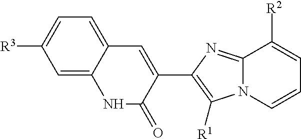

wherein R¹=

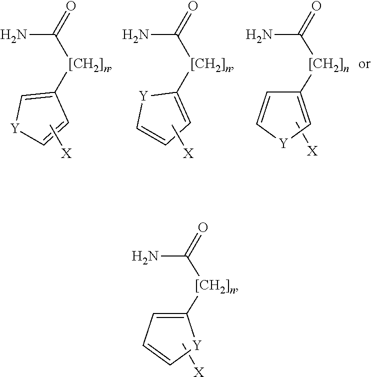

wherein n=1, 2 or 3 and
R²=H or CH₃ and R³=H, OH or OCH₃.

Embodiments include a compound of Formula I:

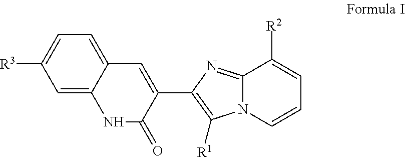

wherein R¹=

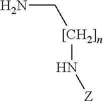

wherein n=1, 2 or 3 and
R²=H or CH₃ and R³=H, OH or OCH₃.

Embodiments include a compound of Formula A:

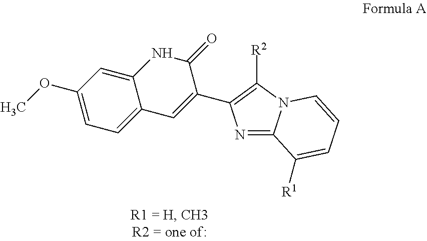

wherein R1=H or CH₃ and R² is one of:

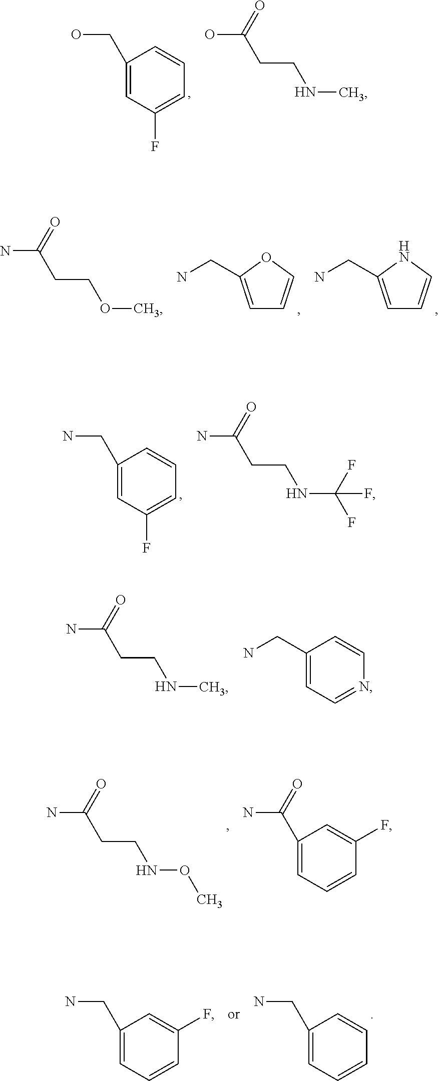

In embodiments include one or more of the compounds described herein can be used therapeutically to treat cancer, as immune checkpoint regulators, modulate PD1 expression, increase a T cell response or increase the activity of an immune cell to treat an ailment, ameliorate one or more side effects of chemotherapy or radiotherapy, prevent or treat radiation induced damage to epithelial cells, treat an infection such as a viral infection and/or reduce inflammation.

Embodiments also include a compound of Formula I or an analog thereof:

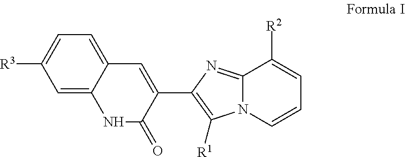

wherein R²=H or CH₃,
wherein R³=H, OH, OCH₃, OCH₂CH₃, CH₂OCH₃,
wherein R¹=an alkyl, an alkyl amine, an ether, an aryl, an aryl halide,
independently selected from the group consisting of hydrogen, amino, amide, F, Cl, Br, I, nitro, alkoxy, hydroxyl, thiol, alkylthio, acyl carboxylic acid, ester, sulfonyl, sulfonamide, —S04H, optionally substituted C1-C20 alkyl, optionally substituted C1-C20 alkenyl, optionally substituted C1-C20 alkynyl, optionally substituted cycloalkyl, optionally substituted cycloalkenyl, optionally substituted heterocycloalkyl, optionally substituted aryl, optionally substituted heteroaryl, optionally substituted aryl halide an optionally substituted phenyl or a functional group selected from:

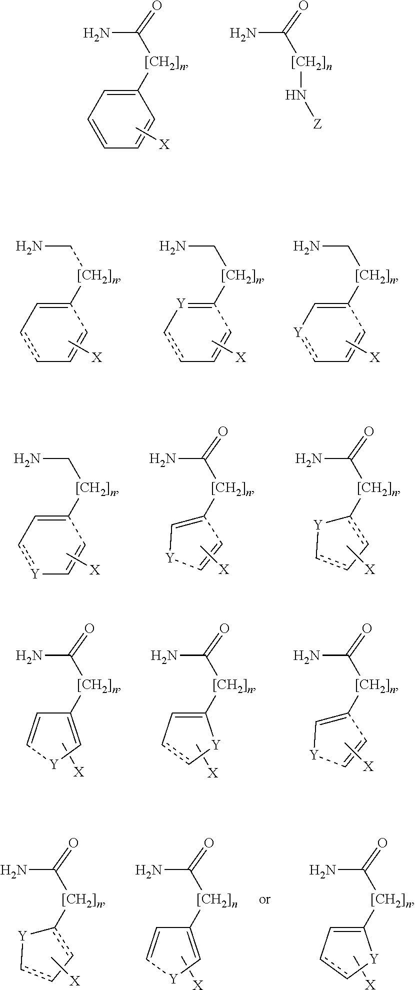

and wherein n=1, 2 or 3,
X=F, CF₃ Cl, OH or CH₂OH,
Y=O, S or N, and
Z=CH₃, CH₂OH or OCH, OCH₂CH₃, CH₂OCH₃.

Embodiments also include a method of treating cancer using a therapeutic amount of the compound of Formula I or an analog. In aspects, the cancer is bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, rectal cancer, skin cancer, blood cancer or testicular cancer, biliary tract cancer, bladder cancer, ganglia cancer (e.g., neuroblastoma), leukemias, lymphoma, liver cancer (e.g., hepatocellular carcinoma), lung cancer (e.g., large cell carcinoma, non-small cell carcinoma and squamous cell carcinoma), soft tissue cancer (e.g., angiosarcoma, leiomyosarcoma, liposarcoma, rhabdomyosarcoma, myxoma and malignant fibrous histiocytoma-pleomorphic sarcoma), stomach cancer or thyroid cancer.

In aspects, the method includes administering one or more additional medicaments to the subject. In aspects, the method includes administration of chemotherapy or radiotherapy to the subject.

Embodiments also include a method of treating one or more side effects of chemotherapy or radiotherapy in a subject. The method can include administering a therapeutically effective amount of a compound of Formula I or an analog.

Embodiments also include a method of preventing or treating radiation induced damage to epithelial cells by administering a therapeutically effective amount of a compound of Formula I or an analog. In aspects, the radiation induced damage to epithelial cells is identified as radiation-induced gastrointestinal syndrome (RIGS), radiation-induced mucositis, radiation-induced oral mucositis, radiation-induced proctitis or radiation-induced enteritis.

Embodiments also include methods of modulating PD-1 expression, increasing a T cell response or increasing activity of an immune cell to treat an ailment. The method can include administering a therapeutic amount of the compound of Formula I or an analog.

Embodiments also include a method of modulating PD-1 expression, increasing a T cell response or increasing the activity of immune cells to treat an ailment. The method can include administering a therapeutic amount of the compound of Formula II, Formula III or Formula IV:

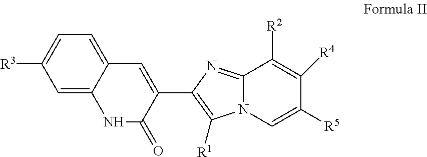

wherein R¹ is an alkyl, alkyl amine, an ether or independently selected from the group consisting of hydrogen, amino, amide, F, Cl, Br, I, nitro, alkoxy, hydroxyl, thiol, alkylthio, acyl carboxylic acid, ester, sulfonyl, sulfonamide, —S0₄H, optionally substituted C1-C20 alkyl, optionally substituted C1-C20 alkenyl, optionally substituted C1-C20 alkynyl, optionally substituted cycloalkyl, optionally substituted cycloalkenyl, optionally substituted heterocycloalkyl, optionally substituted heteroaryl or optionally substituted phenyl,
wherein R³ is H, OH, OCH₃, OCH₂CH₃, CH₂OCH₃, and
wherein R², R⁴ and R⁵ is H or CH₃;

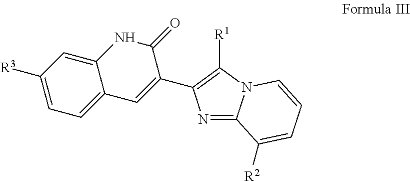

wherein R³ is O—CH₃, R² is CH₃ or H and R¹ is one of

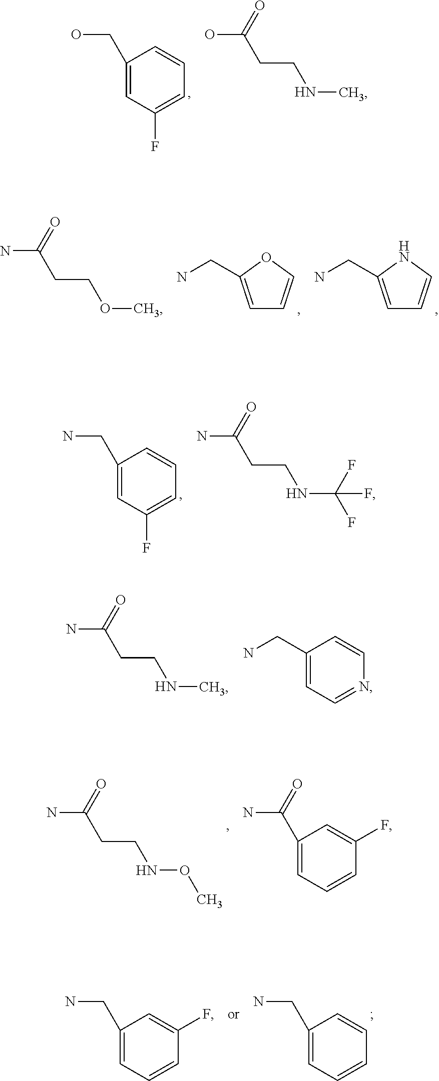

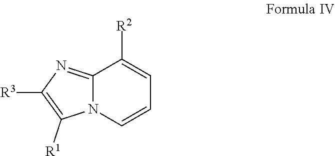

wherein R₃=

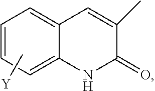

wherein Y=OCH₂CH₃, OCH(n)CH₃, NH₂, OH, OMe, Me, H, CH₂OH, BH₂, SeMe or SMe; and
wherein R₁ and R₂ are independently hydrogen, straight chain or branched C1-C20 alkyl, alkenyl, alkynyl, which is substituted or unsubstituted, cyclo alkyl, cyclo alkenyl, hererocyclic alkyl, or heterocyclic alkenyl, which is substituted or unsubstituted, phenyl, substituted phenyl, aryl, substituted aryl, amino, amido, F, Cl, Br, I, nitro, hydroxyl, thiol, alkylthio, selenol, alkylselenyl, silyl, siloxy, boryl, carboxylic acid, sulfolyl, —SO₄H, alkoxy or acyl groups.

In aspects, the ailment associated with modulating PD-1 expression, increasing a T cell response or increasing the activity of immune cells is cerebral malaria, Trypanosoma cruzi induced myocarditis, influenza virus A, tuberculosis bacillus, a chlamydia lung infection, COPD, acute lung injury, liver infection (HBV or HCV), pancreatitis, Type 1 diabetes, chronic infection, sepsis, an autoimmune disease or HIV.

Embodiments also include a method of inhibiting PD-1, PD-L1 and/or PD-1/PD-L1 interaction. The method can include administering an effective amount of a compound described herein (e.g., Formula II, II or IV) or a pharmaceutically acceptable salt thereof.

Embodiments also include a method of blocking interaction of PD-L1 with PD-1 and/or CD80 in a subject, the method comprising administering an effective amount of the compound described herein (e.g., Formula II, II or IV) or a pharmaceutically acceptable salt thereof.

The above methods (related to PD and PD-1) can include administering one or more additional medicaments, including one or more immune checkpoint regulators. In aspects, the methods include administration of an additional therapeutic agent such as, for example, a small molecule, an antibody, an antibody fragment, an antibody conjugate or an immunomodulating agent.

In aspects, the compounds described above are useful to treat an autoimmune disease such as, for example, Hashimoto thyroiditis and Grave's disease (GD), Rheumatoid arthritis (RA), Systemic Lupus Erythematous (SLE) or Multiple Sclerosis (MS).

TABLE 2

| BCN057 (Formula I) Analogs | Name |
|---|---|
| 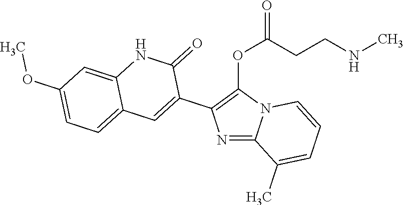 | Formula II |
| 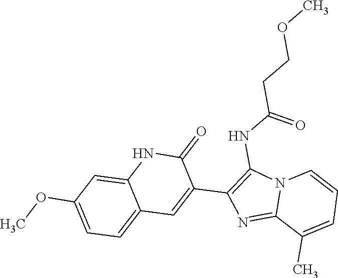 [SMILES: COCCC(=O)Nc1c(-c2cc3ccc(OC)cc3[nH]c2=O)nc2c(C)cccn12] | Formula III |
| 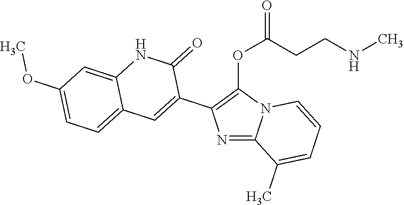 | Formula IV |
| 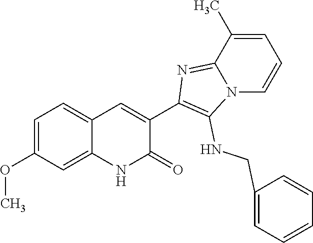 [SMILES: COc1ccc2cc(-c3nc4c(C)cccn4c3NCc3ccccc3)c(=O)[nH]c2c1] | Formula V |
| 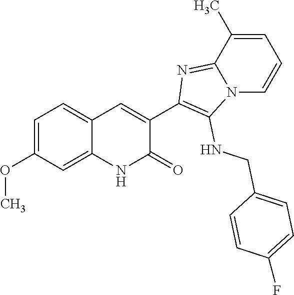 [SMILES: COc1ccc2cc(-c3nc4c(C)cccn4c3NCc3ccc(F)cc3)c(=O)[nH]c2c1] | Formula VI |
| 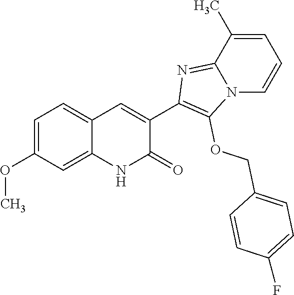 [SMILES: COc1ccc2cc(-c3nc4c(C)cccn4c3OCc3ccc(F)cc3)c(=O)[nH]c2c1] | Formula VII |
| 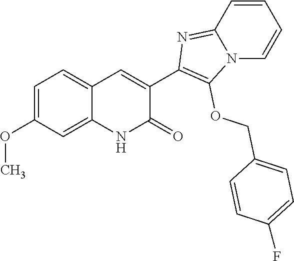 [SMILES: COc1ccc2cc(-c3nc4ccccn4c3OCc3ccc(F)cc3)c(=O)[nH]c2c1] | Formula VIII |
| 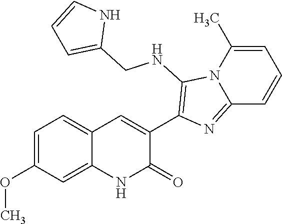 [SMILES: COc1ccc2cc(-c3nc4cccc(C)n4c3NCc3ccc[nH]3)c(=O)[nH]c2c1] | Formula IX |
| 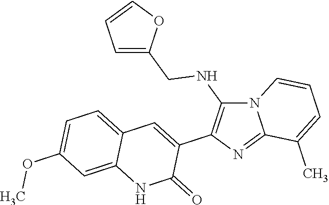 [SMILES: COc1ccc2cc(-c3nc4c(C)cccn4c3NCc3ccco3)c(=O)[nH]c2c1] | Formula X |
| 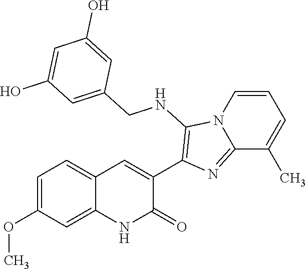 [SMILES: COc1ccc2cc(-c3nc4c(C)cccn4c3NCc3cc(O)cc(O)c3)c(=O)[nH]c2c1] | Formula XI |
| 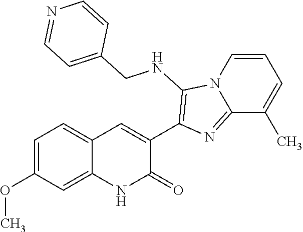 [SMILES: COc1ccc2cc(-c3nc4c(C)cccn4c3NCc3ccncc3)c(=O)[nH]c2c1] | Formula XII |
| 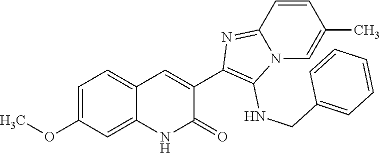 [SMILES: COc1ccc2cc(-c3nc4ccc(C)cn4c3NCc3ccccc3)c(=O)[nH]c2c1] | Formula XIII (Compound H) |
| 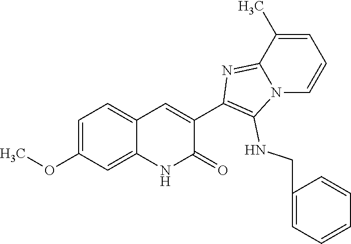 [SMILES: COc1ccc2cc(-c3nc4c(C)cccn4c3NCc3ccccc3)c(=O)[nH]c2c1] | Formula XIV (Compound K) |
| 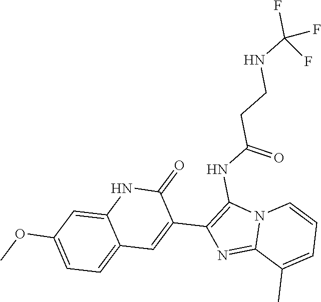 [SMILES: COc1ccc2cc(-c3nc4c(C)cccn4c3NC(=O)CCNC(F)(F)F)c(=O)[nH]c2c1] | Formula XV (Compound BNB-1) |
| 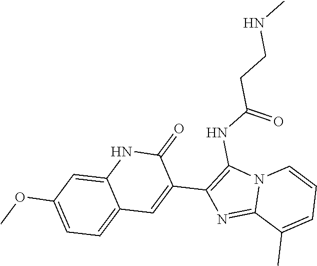 [SMILES: CNCCC(=O)Nc1c(-c2cc3ccc(OC)cc3[nH]c2=O)nc2c(C)cccn12] | Formula XVI (Compound BNB-2) |
| 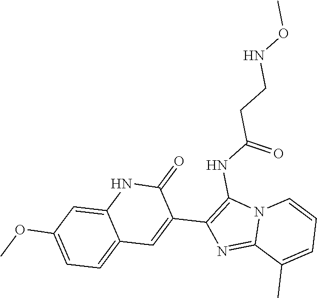 [SMILES: CONCCC(=O)Nc1c(-c2cc3ccc(OC)cc3[nH]c2=O)nc2c(C)cccn12] | Formula XVII (Compound BNB-3) |
| 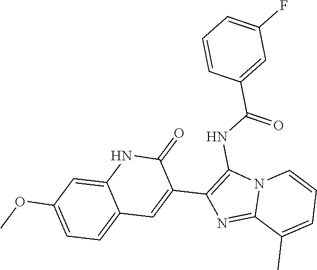 [SMILES: COc1ccc2cc(-c3nc4c(C)cccn4c3NC(=O)c3cccc(F)c3)c(=O)[nH]c2c1] | Formula XVIII (Compound BNB-4) |
| 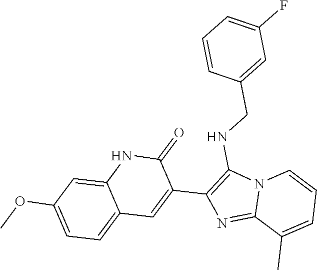 [SMILES: COc1ccc2cc(-c3nc4c(C)cccn4c3NCc3cccc(F)c3)c(=O)[nH]c2c1] | Formula XIX (Compound BNB-5) |
| 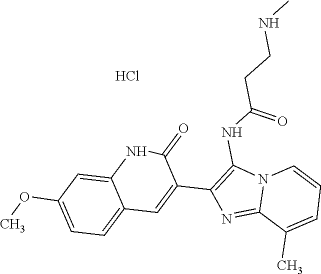 [SMILES: CNCCC(=O)Nc1c(-c2cc3ccc(OC)cc3[nH]c2=O)nc2c(C)cccn12 ; Cl] | BCN076 |
| 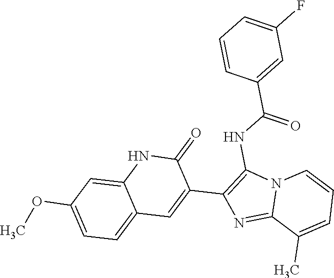 [SMILES: COc1ccc2cc(-c3nc4c(C)cccn4c3NC(=O)c3cccc(F)c3)c(=O)[nH]c2c1] | BCN077 |
| 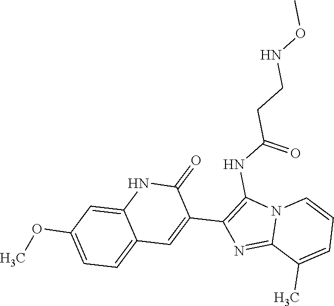 [SMILES: CONCCC(=O)Nc1c(-c2cc3ccc(OC)cc3[nH]c2=O)nc2c(C)cccn12] | BCN036 |

What is claimed is:

1. A compound of Formula I or an analog thereof: 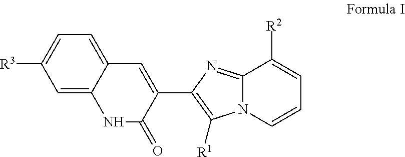
wherein R²=H or CH₃,
wherein R³=H, OH, OCH₃, OCH₂CH₃, CH₂OCH₃,
wherein R¹=an alkyl, an alkyl amine, an ether, an aryl, an aryl halide, independently selected from the group consisting of hydrogen, amino, amide, F, Cl, Br, I, nitro, alkoxy, hydroxyl, thiol, alkylthio, acyl carboxylic acid, ester, sulfonyl, sulfonamide, —S04H, optionally substituted C1-C20 alkyl, optionally substituted C1-C20 alkenyl, optionally substituted C1-C20 alkynyl, optionally substituted cycloalkyl, optionally substituted cycloalkenyl, optionally substituted heterocycloalkyl, optionally substituted aryl, optionally substituted heteroaryl, optionally substituted aryl halide an optionally substituted phenyl or a functional group selected from: 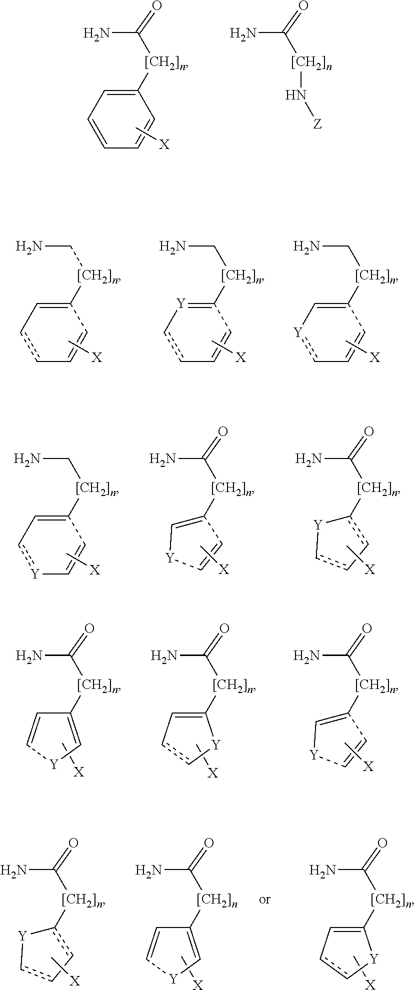
and wherein n=1, 2 or 3,
X=F, CFs Cl, OH or CH₂OH,
Y=O, S or N, and
Z=CH₃, CH₂OH or OCH, OCH₂CH₃, CH₂OCH₃.

2. A method of treating cancer comprising administering a therapeutic amount of a compound of claim 1

3. The method of claim 2, wherein the cancer is bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, rectal cancer, skin cancer, blood cancer or testicular cancer, biliary tract cancer, bladder cancer, ganglia cancer (e.g., neuroblastoma), leukemias, lymphoma, liver cancer (e.g., hepatocellular carcinoma), lung cancer (e.g., large cell carcinoma, non-small cell carcinoma and squamous cell carcinoma), soft tissue cancer (e.g., angiosarcoma, leiomyosarcoma, liposarcoma, rhabdomyosarcoma, myxoma and malignant fibrous histiocytoma-pleomorphic sarcoma), stomach cancer or thyroid cancer.

4. The method of claim 2, wherein the method further comprises administering one or more additional therapeutic agents, a chemotherapeutic or a radiotherapeutic to the subject.

5. The method of claim 4, wherein the wherein the one or more additional therapeutic agents is a small molecule, an antibody, an antibody fragment, an antibody conjugate or an immunomodulating agent.

6. A method of treating one or more side effects of chemotherapy or radiotherapy in a subject in need thereof, the method comprising administering a therapeutically effective amount of a compound of claim 1.

7. A method of preventing or treating radiation induced damage to epithelial cells in a subject in need thereof, the method comprising administering a therapeutically effective amount of a compound of claim 1.

8. The method of claim 7, wherein the radiation induced damage to epithelial cells is identified as one or more of radiation-induced gastrointestinal syndrome (RIGS), radiation-induced mucositis, radiation-induced oral mucositis, radiation-induced proctitis and radiation-induced enteritis.

9. A method of modulating PD-1 expression, increasing a T cell response or increasing activity of an immune cell to treat an ailment, the method comprising administering a therapeutic amount of the compound of claim 1 to a patient in need thereof.

10. A method blocking interaction of PD-L1 with PD-1 and/or CD80 to treat an ailment, the method comprising administering an effective amount of a compound of claim 1, or a pharmaceutically acceptable salt thereof, to a subject in need thereof.

11. The method of claim 9, wherein the ailment is at least one of cerebral malaria, Trypanosoma cruzi induced myocarditis, influenza virus A, tuberculosis bacillus, a chlamydia lung infection, COPD, acute lung injury, liver infection (HBV or HCV), pancreatitis, Type 1 diabetes, chronic infection, sepsis, an autoimmune disease or HIV.

12. The method of claim 11, wherein the autoimmune disease is at least one of Hashimoto thyroiditis and Grave's disease (GD), Rheumatoid arthritis (RA), Systemic Lupus Erythematous (SLE) or Multiple Sclerosis (MS).

13. A method of treating an ailment, the method comprising administering a therapeutic amount of the compound of Formula II, Formula III or Formula IV to a patient in need thereof: 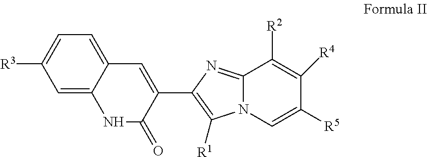
wherein R¹ is an alkyl, alkyl amine, an ether or independently selected from the group consisting of hydrogen, amino, amide, F, Cl, Br, I, nitro, alkoxy, hydroxyl, thiol, alkylthio, acyl carboxylic acid, ester, sulfonyl, sulfonamide, —S0₄H, optionally substituted C1-C20 alkyl, optionally substituted C1-C20 alkenyl, optionally substituted C1-C20 alkynyl, optionally substituted cycloalkyl, optionally substituted cycloalkenyl, optionally substituted heterocycloalkyl, optionally substituted heteroaryl or optionally substituted phenyl,
wherein R³ is H, OH, OCH₃, OCH₂CH₃, CH₂OCH₃, and
wherein R², R⁴ and R⁵ is H or CH₃; 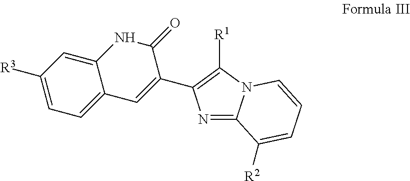
wherein R³ is O—CH₃, R² is CH₃ or H and R¹ is one of 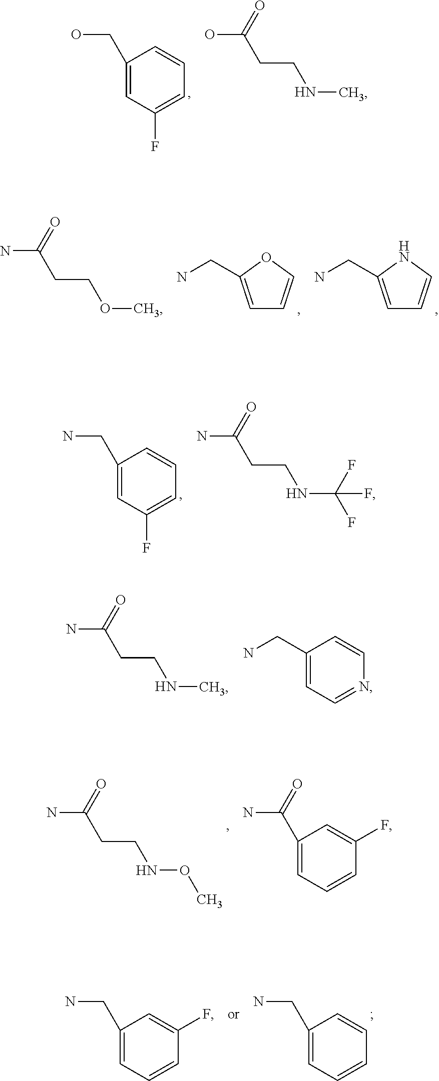 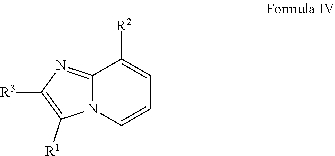
wherein R₃= 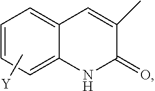
wherein Y=OCH₂CH₃, OCH(n)CH₃, NH₂, OH, OCH₃, CH₃, H, CH₂OH, BH₂, SeCH₃ or SCH₃; and
wherein R₁ and R₂ are independently hydrogen, straight chain or branched C1-C20 alkyl, alkenyl, alkynyl, which is substituted or unsubstituted, cyclo alkyl, cyclo alkenyl, hererocyclic alkyl, or heterocyclic alkenyl, which is substituted or unsubstituted, phenyl, substituted phenyl, aryl, substituted aryl, amino, amido, F, Cl, Br, I, nitro, hydroxyl, thiol, alkylthio, selenol, alkylselenyl, silyl, siloxy, boryl, carboxylic acid, sulfolyl, —SO₄H, alkoxy or acyl groups.

14. The method of claim 13, wherein the ailment is at least one of cerebral malaria, Trypanosoma cruzi induced myocarditis, influenza virus A, tuberculosis bacillus, a chlamydia lung infection, COPD, acute lung injury, liver infection (HBV or HCV), pancreatitis, Type 1 diabetes, chronic infection, sepsis, an autoimmune disease or HIV.

15. The method of claim 14, wherein the autoimmune disease is at least one of Hashimoto thyroiditis and Grave's disease (GD), Rheumatoid arthritis (RA), Systemic Lupus Erythematous (SLE) or Multiple Sclerosis (MS).

16. A method of inhibiting PD-1, PD-L1 and/or PD-1/PD-L1 interaction to treat an ailment, the method comprising administering an effective amount of a compound of claim 13 (Formula II, Formula III or Formula IV), or a pharmaceutically acceptable salt thereof, to a patient in need thereof.

17. A method blocking interaction of PD-L1 with PD-1 and/or CD80 to treat an ailment, the method comprising administering an effective amount of the compound of claim 13 (Formula II, Formula III or Formula IV), or a pharmaceutically acceptable salt thereof, to a patient in need thereof.

18. The method of claim 13, wherein the method further comprises administering one or more additional therapeutic agents, a chemotherapeutic or a radiotherapeutic to the subject.

19. The method of claim 18, wherein the wherein the one or more additional therapeutic agents is an immune checkpoint regulator, a small molecule, an antibody, an antibody fragment, an antibody conjugate or an immunomodulating agent.

20. A method of inhibiting LAG-3 expression to treat an ailment, the method comprising administering an effective amount of the compound of claim 13 (Formula II, Formula III or Formula IV), or a pharmaceutically acceptable salt thereof, to a patient in need thereof.
